(19) United States
(12) Patent Application Publication
(10) Pub. No.: US 2008/0280850 A1
(43) Pub. Date: Nov. 13, 2008

(54) Methods and Compositions for Treating Flaviviruses, Pestiviruses and Hepacivirus

(76) Inventors: Jean-Pierre Sommadossi, Cambridge, MA (US); Gilles Gosselin, Montpellier (FR); Richard Storer, Kent (GB); James Egan, Kensington, MA (US)

Correspondence Address: JONES DAY, 222 EAST 41ST ST, NEW YORK, NY, 10017, US

(21) Appl. No.: 11/884,414

(22) PCT Filed: Sep. 26, 2005
(86) PCT No.: PCT/US2005/034786

§ 371 Date: Jan. 15, 2008

Related U.S. Application Data
(60) Provisional application No. 60/613,085, filed on Sep. 24, 2004.

(51) Int. Cl. C07H 19/10; C07H 19/06; C07H 19/04; A61K 31/7068; A61K 31/706; A61P 31/12

(52) U.S. Cl. 514/49; 536/28.4; 536/28.1; 536/26.26; 536/26.1; 514/43; 514/52

(57) ABSTRACT

A method and composition for treating a host infected with flavivirus, pestivirus or hepacivirus comprising administering an effective flavivirus, pestivirus or hepacivirus treatment amount of a described base-modified nucleoside or a pharmaceutically acceptable salt or prodrug thereof, is provided.

### CROSS-REFERENCE TO RELATED APPLICATIONS

This application claims priority to U.S. Provisional Application No. 60/613,085, filed Sep. 24, 2004.

### FIELD OF THE INVENTION

This invention is in the area of pharmaceutical chemistry, and in particular, is a compound, method and composition for the treatment of flaviviruses, pestiviruses and hepaciviruses, and in particular for hepatitis C virus infection.

### BACKGROUND OF THE INVENTION

Pestiviruses and flaviviruses belong to the Flaviviridae family of viruses along with hepatitis C virus. The pestivirus genus includes bovine viral diarrhea virus (BVDV), classical swine fever virus (CSFV, also called hog cholera virus) and border disease virus (BDV) of sheep (Moennig, V. et al. Adv. Vir. Res. 1992, 41, 53-98). Pestivirus infections of domesticated livestock (cattle, pigs and sheep) cause significant economic losses worldwide. BVDV causes mucosal disease in cattle and is of significant economic importance to the livestock industry (Meyers, G. and Thiel, H.-J., Advances in Virus Research, 1996, 47, 53-118; Moennig V., et al, Adv. Vir. Res. 1992, 41, 53-98).

Human pestiviruses have not been as extensively characterized as the animal pestiviruses. However, serological surveys indicate considerable pestivirus exposure in humans. Pestivirus infections in man have been implicated in several diseases including congenital brain injury, infantile gastroenteritis and chronic diarrhea in human immunodeficiency virus (HIV) positive patients. M. Giangaspero et al., Arch. Virol. Suppl., 1993, 7, 53-62; M. Giangaspero et al., Int. J. Std. Aids, 1993, 4 (5): 300-302.

The flavivirus genus includes more than 68 members separated into groups on the basis of serological relatedness (Calisher et al., J. Gen. Virol, 1993, 70, 37-43). Clinical symptoms vary and include fever, encephalitis and hemorrhagic fever. Fields Virology, Editors: Fields, B. N., Knipe, D. M., and Howley, P. M., Lippincott-Raven Publishers, Philadelphia, Pa., 1996, Chapter 31, 931-959. Flaviviruses of global concern that are associated with human disease include the dengue hemorrhagic fever viruses (DHF), yellow fever virus, shock syndrome and Japanese encephalitis virus. Halstead, S. B., Rev. Infect. Dis., 1984, 6, 251-264; Halstead, S. B., Science, 239:476-481, 1988; Monath, T. P., New Eng. J. Med., 1988, 319, 641-643.

Pestiviruses and hepaciviruses are closely related virus groups within the Flaviviridae family. Other closely related viruses in this family include the GB virus A, GB virus A-like agents, GB virus-B and GB virus-C (also called hepatitis G virus, HGV). The hepacivirus group (hepatitis C virus; HCV) consists of a number of closely related but genotypically distinguishable viruses that infect humans. There are approximately 6 HCV genotypes and more than 50 subtypes. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

The hepatitis C virus (HCV) is the leading cause of chronic liver disease worldwide. (Boyer, N. et al. J. Hepatol. 32:98-112, 2000). HCV causes a slow growing viral infection and is the major cause of cirrhosis and hepatocellular carcinoma (Di Besceglie, A. M. and Bacon, B. R., Scientific American, October: 80-85, (1999); Boyer, N. et al. J. Hepatol. 32:98-112, 2000). An estimated 170 million persons are infected with HCV worldwide. (Boyer, N. et al. J Hepatol. 32:98-112, 2000). Cirrhosis caused by chronic hepatitis C infection accounts for 8,000-12,000 deaths per year in the United States, and HCV infection is the leading indication for liver transplantation.

HCV is known to cause at least 80% of posttransfusion hepatitis and a substantial proportion of sporadic acute hepatitis. Preliminary evidence also implicates HCV in many cases of “idiopathic” chronic hepatitis, “cryptogenic” cirrhosis, and probably hepatocellular carcinoma unrelated to other hepatitis viruses, such as Hepatitis B Virus (HBV). A small proportion of healthy persons appear to be chronic HCV carriers, varying with geography and other epidemiological factors. The numbers may substantially exceed those for HBV, though information is still preliminary; how many of these persons have subclinical chronic liver disease is unclear. (The Merck Manual, ch. 69, p. 901, 16th ed., (1992)).

HCV is an enveloped virus containing a positive-sense single-stranded RNA genome of approximately 9.4 kb. The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

A significant focus of current antiviral research is directed to the development of improved methods of treatment of chronic HCV infections in humans (Di Besceglie, A. M. and Bacon, B. R., Scientific American, October: 80-85, (1999)).

In view of the severity of diseases associated with pestiviruses and flaviviruses, and their pervasiveness in animal and man, it is an object of the present invention to provide a compound, method and composition for the treatment of a host infected with flavivirus, pestivirus or hepacivirus.

### SUMMARY OF THE INVENTION

Compounds, methods and compositions for the treatment of a host infected with a flavivirus, pestivirus or hepacivirus infection are described that includes an effective treatment amount of a modified nucleoside of the Formulas (I)-(VI), or a pharmaceutically acceptable salt or prodrug thereof. In one embodiment, the virus is hepatitis C.

In summary, the present invention includes the following features:
(a) Modified nucleosides of Formula (I)-(VI), and pharmaceutically acceptable salts, esters and prodrugs thereof;
(b) Modified nucleosides of Formula (I)-(VI), and pharmaceutically acceptable salts, esters and prodrugs thereof for use in the treatment or prophylaxis of a flavivirus, pestivirus or hepacivirus infection, especially in individuals diagnosed as having a flavivirus, pestivirus or hepacivirus infection or being at risk for becoming infected by flavivirus, pestivirus or hepacivirus;
(c) use of these modified nucleosides of Formula (I)-(VI), and pharmaceutically acceptable salts, esters and prodrugs thereof in the manufacture of a medicament for treatment of a flavivirus, pestivirus or hepacivirus infection;
(d) pharmaceutical formulations comprising the modified nucleosides of Formula (I)-(VI), and pharmaceutically acceptable salts, esters and prodrugs thereof together with a pharmaceutically acceptable carrier or diluent;
(e) modified nucleosides of Formula (I)-(VI), and pharmaceutically acceptable salts, esters and prodrugs substantially in the absence of enantiomers of the described nucleoside, or substantially isolated from other chemical entities;
(f) processes for the preparation of modified nucleosides of Formula (I)-(VI), and pharmaceutically acceptable salts, esters and prodrugs; and
(g) processes for the preparation of modified nucleosides of Formula (I)-(VI), and pharmaceutically acceptable salts, esters and prodrugs substantially in the absence of enantiomers of the described nucleoside, or substantially isolated from other chemical entities.

In a first principal embodiment, a method for the treatment of a host infected with a flavivirus, pestivirus or hepacivirus, and in particular HCV, infection is provided, comprising administering an effective treatment amount of a compound of Formula (I) or (II):

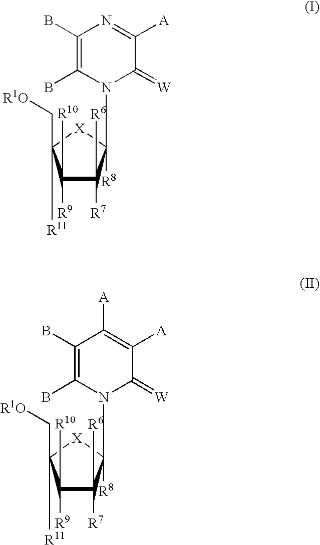

or a pharmaceutically acceptable salt or prodrug thereof, wherein:
R¹ is independently H, optionally substituted alkyl (including lower alkyl); acyl (including lower acyl); phosphate (including mono-, di- or triphosphate or a stabilized phosphate prodrug); sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹ is independently H or phosphate (including mono-, di- or triphosphate);
each A is independently a straight, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, 5H, —S-alkyl, —S-acyl, —S-aryl, —S-cycloalkyl, —S-aralkyl, F, Cl, Br, I, —CO₂-alkyl, —CONH-alkyl, —CON-dialkyl, CF₃, —CHₘOH, —(CH₂)ₘNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄alkoxycarbonyl, N₃ or C₁₋₆ alkoxy;
each B is independently H, a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, —CO—O-alkoxyalkyl, —CONHR⁴, —C(NR⁴)N(R⁴)₂, —C(S)N(R⁴)₂, —CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, SH, —S-alkyl, —S-acyl, —S-aryl, —S-cycloalkyl, —S-aralkyl, F, Cl, Br, I, —CO₂-alkyl, —CONH-alkyl, —CON-dialkyl, CF₃, —CHₘOH, —(CH₂)ₙNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each Y³ is independently H, F, Cl, Br or I;
each R⁴ and R⁵ is independently hydrogen, acyl (including lower acyl), alkyl (including but not limited to methyl, ethyl, propyl and cyclopropyl), lower alkyl, alkenyl, alkynyl or cycloalkyl.

### X is O or CH;

each R⁶ is independently an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, (CH₂)ₘC(O)N(R⁴)₂, C(O)OR⁴ or cyano;
each R⁷ is independently OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴, (CH₂)ₘC(O)SR⁴—(CH₂)ₘC(O)NHR⁴, (CH₂)ₘC(O)N(R⁴)₂, —C(O)OR⁴, —C(O)SR⁴, —O(R⁴), S(R⁴), NO₂, —NR⁴R⁵, azido, cyano, SCN, OCN, NCO or halo;
each R⁸ and R¹¹ is independently hydrogen, an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, haloalkenyl, Br-vinyl, optionally substituted alkynyl, haloalkynyl, —CH₂C(O)N(R⁴)₂, —(CH₂)ₘC(O)OR⁴, —(CH₂)ₘC(O)NHR⁴, —C(O)OR⁴, cyano, NH-acyl or N(acyl)₂;
each R⁹ and R¹⁰ are independently hydrogen, OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, NO₂, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴—(CH₂)ₘC(O)SR⁴, (CH₂)ₘC(O)NHR⁴, (CH₂)ₘC(O)N(R⁴)₂, —C(O)OR⁴, —C(O)SR⁴, —O(R⁴), —O(aralkyl), —S(R⁴), NO₂, —NR⁴R⁵, —NH(aralkyl), azido, cyano, SCN, OCN, NCO or halo;
each m is independently 1 or 2; and
alternatively, R⁶ and R¹⁰, R⁷ and R⁹, R⁸ and R⁷ or R⁹ and R¹¹ can come together to form a bridged compound selected from the group consisting of optionally substituted carbocycle (typically a 3-7 membered carbocyclic ring) or optionally substituted heterocycle (typically a 3-7 membered heterocyclic ring having one or more O, S and/or N); or
alternatively, R⁶ and R⁷ or R⁹ and R¹⁰ can come together to form a spiro compound selected from the group consisting of optionally substituted carbocycle (typically a 3-7 membered carbocyclic ring) or optionally substituted heterocycle (typically a 3-7 membered heterocyclic ring having one or more O, S and/or N); and
each W is independently O, S or CH.

In another principal embodiment, a method for the treatment of a host infected with a flavivirus, pestivirus or hepacivirus, and in particular HCV, infection is provided, comprising administering an effective treatment amount of a compound of Formula (III), (IV) or (V):

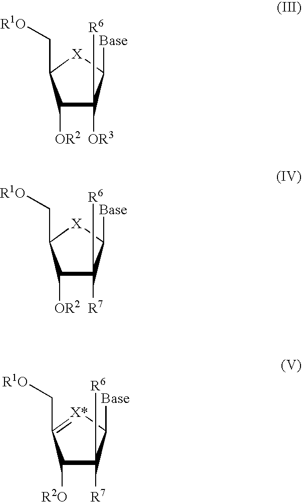

or a pharmaceutically acceptable salt or prodrug thereof, wherein
R¹, R² and R³ are each independently H, optionally substituted alkyl (including lower alkyl); acyl (including lower acyl); phosphate (including mono-, di- or triphosphate or a stabilized phosphate prodrug); sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹, R² or R³ is independently H or phosphate (including mono-, di- or triphosphate); wherein in one embodiment R² and/or R³ is not phosphate (including mono-, di- or triphosphate or a stabilized phosphate prodrug);
each R⁶ is independently H, OH, NO₂, halo, azido, alkenyl and alkynyl an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, —(CH₂)ₘC(O)N(R⁴)₂, C(O)OR⁴ or cyano;
X and X* are independently O or CH;
each R⁷ is independently OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴, —(CH₂)ₘC(O)SR⁴—(CH₂)ₘC(O)NHR⁴, (CH₂)ₘC(O)N(R⁴)₂, —C(O)OR⁴, —C(O)SR⁴, —O(R⁴), S(4), NO₂, —NR⁴R⁵, azido, cyano, SCN, OCN, NCO or halo; and
alternatively, R⁶ and R⁷ can come together to form a spiro compound selected from the group consisting of optionally substituted carbocycle (typically a 3-7 membered carbocyclic ring) or optionally substituted heterocycle (typically a 3-7 membered heterocyclic ring having one or more O, S and/or N);
each m is independently 1 or 2;
and Base is independently

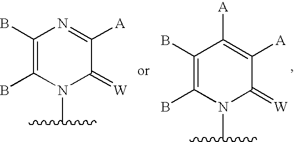

wherein A, B and W are as defined above.

In another principal embodiment, a method for the treatment of a host infected with a flavivirus, pestivirus or hepacivirus, and in particular HCV, infection is provided, comprising administering an effective treatment amount of a compound of compound of Formula (VI):

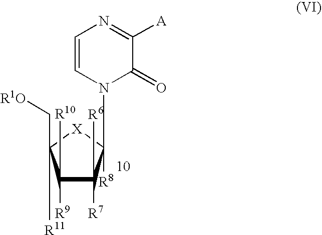

wherein A, R¹, R⁶, R⁷, R⁸, R⁹, R¹⁰ and R¹¹ are as defined above.

The modified nucleosides of this invention may inhibit flavivirus, pestivirus or hepacivirus polymerase activity. These nucleosides can be assessed for their ability to inhibit flavivirus, pestivirus or hepacivirus polymerase activity in vitro according to standard screening methods. In one embodiment the efficacy of the anti-flavivirus, pestivirus or hepacivirus compound is measured according to the concentration of compound necessary to reduce the plaque number of the virus in vitro by 50% (i.e. the compound's EC₅₀). In a preferred embodiment, the compound exhibits an EC₅₀ of less than 15 or typically, less than 10 micromolar in vitro.

In another embodiment, the active compound can be administered in combination or alternation with another anti-flavivirus, pestivirus or hepacivirus agent. A variety of known antiviral agents can be used in combination or alternation with the compounds of the invention. In combination therapy, effective dosages of two or more agents are administered together, whereas during alternation therapy an effective dosage of each agent is administered serially.

HCV is a member of the Flaviviridae family; however, now, HCV has been placed in a new monotypic genus, hepacivirus. Therefore, in one embodiment, the flavivirus or pestivirus is not HCV. However, in a separate embodiment, the virus is a hepacivirus, and in one embodiment, is HCV.

### DETAILED DESCRIPTION OF THE INVENTION

The invention is a compound, method and composition for the treatment of flavivirus, pestivirus or hepacivirus, and in particular HCV, infection in humans and other host animals, that includes the administration of an effective flavivirus, pestivirus or hepacivirus treatment amount of an modified nucleoside as described herein or a pharmaceutically acceptable salt or prodrug thereof, optionally in a pharmaceutically acceptable carrier. The compounds of this invention either possess antiviral (i.e., flavivirus, pestivirus or hepacivirus, and in particular HCV) activity, or are metabolized to a compound that exhibits such activity.

### I. ACTIVE COMPOUND

In a first principal embodiment, a compound is provided for the treatment of a host infected with a flavivirus, pestivirus or hepacivirus, and in particular HCV, of Formula (I):

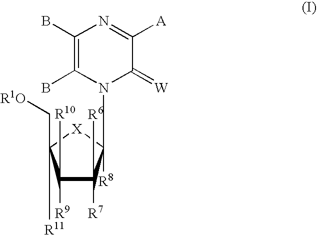

or a pharmaceutically acceptable salt or prodrug thereof, wherein:
R¹ is independently H, optionally substituted alkyl (including lower alkyl); acyl (including lower acyl); phosphate (including mono-, di- or triphosphate or a stabilized phosphate prodrug); sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹ is independently H or phosphate (including mono-, di- or triphosphate);
each A is independently a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, C(═S)NH₂, —C(═NH)NH₂, —C-(5-membered heterocycle) having one or more O, S or N atoms, —C-imidazole; cycloalkyl, acyl, Br-vinyl, —O-alkyl, O-alkenyl, O-alkynyl, O-aryl, O-aralkyl, —O-acyl, O-cycloalkyl, NH-alkyl, N-dialkyl, NH-acyl, N-aryl, N-aralkyl, NH-cycloalkyl, SH, S-alkyl, S-acyl, S-aryl, S-cycloalkyl, S-aralkyl, F, Cl, Br, I, CO₂-alkyl, CONH-alkyl, CON-dialkyl, CF₃, CHₘOH, (CH₂)ₘNH₂, (CH₂)ₘC(O)OH, (CH₂)ₘCN, (CH₂)ₘNO₂ (CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each B is independently H, a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, C(═S)NH₂, —C(═NH)NH₂, —C-(5-membered heterocycle) having one or more O, S or N atoms, —C-imidazole; cycloalkyl, acyl, Br-vinyl, —O-alkyl, O-alkenyl, O-alkynyl, O-aryl, O-aralkyl, —O-acyl, O-cycloalkyl, NH-alkyl, N-dialkyl, NH-acyl, N-aryl, N-aralkyl, NH-cycloalkyl, SH, S-alkyl, S-acyl, S-aryl, S-cycloalkyl, S-aralkyl, F, Cl, Br, I, CO₂-alkyl, CONH-alkyl, CON-dialkyl, CF₃, CHₘOH, (CH₂)ₘNH₂, (CH₂)ₘC(O)OH, (CH₂)ₘCN, (CH₂)ₘNO₂ (CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each Y³ is independently H, F, Cl, Br or I;
each R⁴ and R⁵ is independently hydrogen, acyl (including lower acyl), alkyl (including but not limited to methyl, ethyl, propyl and cyclopropyl), lower alkyl, alkenyl, alkynyl or cycloalkyl.

### X is O or CH;

each R⁶ is independently an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, —(CH₂)ₘC(O)N(R⁴)₂, C(O)OR⁴ or cyano;
each R⁷ is independently OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴, —(CH₂)ₘC(O)SR⁴—(CH₂)ₘC(O)NHR⁴, —(CH₂)ₘC(O)N(R⁴)₂, —C(O)OR⁴, —C(O)SR⁴, —O(R⁴), S(R⁴), NO₂, —NR⁴R⁵, azido, cyano, SCN, OCN, NCO or halo;
each R⁸ and R¹¹ is independently hydrogen, an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, haloalkenyl, Br-vinyl, optionally substituted alkynyl, haloalkynyl, —CH₂C(O)N(R⁴)₂, —(CH₂)ₘC(O)OR⁴, —(CH₂)ₘC(O)NHR⁴, —C(O)OR⁴, cyano, NH-acyl or N(acyl)₂;
each R⁹ and R¹⁰ are independently hydrogen, OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, NO₂, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴—(CH₂)ₘC(O)SR⁴, —(CH₂)ₘC(O)NHR⁴, (CH₂)ₘC(O)N(R⁴)₂, —C(O)OR⁴, —C(O)SR⁴, —O(R⁴), —O(aralkyl), —S(R⁴), NO₂, —NR⁴R⁵, —NH(aralkyl), azido, cyano, SCN, OCN, NCO or halo;
each m is independently 1 or 2; and
alternatively, R⁶ and R¹⁰, R⁷ and R⁹, R⁸ and R⁷ or R⁹ and R¹¹ can come together to form a bridged compound selected from the group consisting of optionally substituted carbocycle (typically a 3-7 membered carbocyclic ring) or optionally substituted heterocycle (typically a 3-7 membered heterocyclic ring having one or more O, S and/or N); or
alternatively, R⁶ and R⁷ or R⁹ and R¹⁰ can come together to form a spiro compound selected from the group consisting of optionally substituted carbocycle (typically a 3-7 membered carbocyclic ring) or optionally substituted heterocycle (typically a 3-7 membered heterocyclic ring having one or more O, S and/or N); and

### W is O, S or CH.

In one subembodiment, the compound is provided for the treatment of a host infected with a flavivirus, pestivirus or hepacivirus, and in particular HCV, of Formula (I) or a pharmaceutically acceptable salt or prodrug thereof, is provided wherein:

R¹ is independently H, optionally substituted alkyl; acyl; phosphate; a lipid, including a phospholipid; an amino acid; a carbohydrate; a peptide; cholesterol; or other pharmaceutically acceptable leaving group which when administered in vivo is capable of providing a compound wherein R¹ is independently H or phosphate;
A is independently a straight chained, branched or cyclic optionally substituted alkyl, CH₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, —C(—S)NH₂, —C(═NH)NH₂, —C-(5-membered heterocycle) having one or more O, S or N atoms; acyl, CONH-alkyl, CON-dialkyl, (CH₂)ₘC(O)OH, (CH₂)ₘCN, (CH₂)ₘNO₂ (CH₂)ₘCONH₂;
each B is independently H, a straight chained, branched or cyclic optionally substituted alkyl, CH₃, CF₃, C(Y³)₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, OH, OR⁴, NH₂, NHR⁴, NR⁴R⁵, SH or SR⁵, CF₃, CHₘOH, (CH₂)ₘNH₂, (CH₂)ₘC(O)OH, (CH₂)ₘCN, (CH₂)ₘNO₂ (CH₂)ₘCONH₂, C₁₋₄ alkylamino, or C₁₋₆ alkoxy;
each Y³ is independently H, F, Cl, Br or I;
each R⁴ and R⁵ is independently hydrogen, acyl, alkyl, alkenyl, alkynyl or cycloalkyl.

### X is O;

each R⁶ is independently an optionally substituted alkyl, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, or cyano;
each R⁷ is independently OH, OR², optionally substituted alkyl, or halo;
each R⁸ and R¹¹ is independently hydrogen, an optionally substituted alkyl;
each R⁹ and R¹⁰ are independently hydrogen, OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, NO₂, haloalkenyl, Br-vinyl, optionally substituted alkynyl, haloalkynyl, optionally substituted carbocycle, optionally substituted heterocycle (typically a 3-7 membered heterocyclic ring having one or more O, S and/or N), optionally substituted heteroaryl, —(CH₂)ₘC(O)OR⁴—(CH₂)ₘC(O)SR⁴, —(CH₂)ₘC(O)NHR⁴, —(CH₂)ₘC(O)N(R⁴)₂, —C(O)OR⁴, —C(O)SR⁴, —O(R⁴), —O(aralkyl), —S(R⁴), NO₂, —NR⁴R⁵, —NH(aralkyl), azido, cyano, SCN, OCN, NCO or halo;
each m is independently 1 or 2.

In one subembodiment, X is O, each B is independently H or a straight chained, branched or cyclic optionally substituted alkyl or a halogen (Cl, Br, I, F), each R⁷ and R⁹ is independently OH or OR² and R¹ is H or phosphate.

In another subembodiment, the compound of Formula (I) or a pharmaceutically acceptable salt or prodrug thereof, is provided wherein:

R¹ is H or phosphate;

### A is CONHR⁴; and

### B is H.

In a second principal embodiment, a compound is provided for the treatment of a host infected with a flavivirus, pestivirus or hepacivirus, and in particular HCV, of Formula (II):

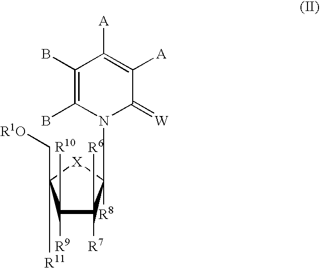

or a pharmaceutically acceptable salt or prodrug thereof, wherein R¹, R², R³, R⁴, R⁵, R⁶, R⁷, R⁸, R⁹, R¹⁰, R¹¹, A, B and W are as defined above.

In one subembodiment, the compound of Formula (II), or a pharmaceutically acceptable salt or prodrug thereof, is provided wherein:

R¹ is H or phosphate;
each A is independently H, CH₃, CF₃ or CH₂CH₃.

In a third principal embodiment, a compound is provided for the treatment of a host infected with a flavivirus, pestivirus or hepacivirus, and in particular HCV, of Formula (III), (IV) or (V):

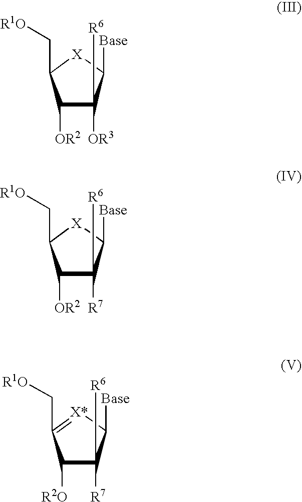

or a pharmaceutically acceptable salt or prodrug thereof, wherein
R¹, R² and R³ are independently H, optionally substituted alkyl (including lower alkyl); acyl (including lower acyl); phosphate (including mono-, di- or triphosphate or a stabilized phosphate prodrug); sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹, R² or R³ is independently H or phosphate (including mono-, di- or triphosphate); wherein in one embodiment R² and/or R³ is not phosphate (including mono-, di- or triphosphate or a stabilized phosphate prodrug);
each R⁶ is independently H, OH, NO₂, halo, azido, alkenyl and alkynyl an optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, (CH₂)ₘC(O)N(R⁴)₂, C(O)OR⁴ or cyano;
X and X* are independently O or CH;
each R⁷ is independently OH, OR², optionally substituted alkyl (including lower alkyl), CH₃, CH₂CN, CH₂N₃, CH₂NH₂, CH₂NHCH₃, CH₂N(CH₃)₂, CH₂OH, halogenated alkyl (including halogenated lower alkyl), CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle (typically a 3-7 membered carbocyclic ring), optionally substituted heterocycle (typically a 3-7 membered heterocyclic ring having one or more O, S and/or N), optionally substituted heteroaryl (typically a 3-7 membered heteroaromatic ring having one or more O, S and/or N), —(CH₂)ₘC(O)OR⁴, —(CH₂)ₘC(O)SR⁴—(CH₂)ₘC(O)NHR⁴, (CH₂)ₘC(O)N(R⁴)₂, —C(O)OR⁴, —C(O)SR⁴, —O(R⁴), S(R⁴), NO₂, —NR⁴R⁵, azido, cyano, SCN, OCN, NCO or halo; and
alternatively, R⁶ and R⁷ can come together to form a spiro compound selected from the group consisting of optionally substituted carbocycle (typically a 3-7 membered carbocyclic ring) or optionally substituted heterocycle (typically a 3-7 membered heterocyclic ring having one or more O, S and/or N);
each m is independently 1 or 2;
and Base is independently:

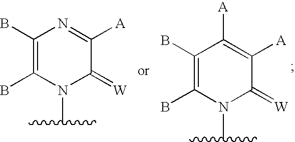

wherein A, B and W are as defined above.

In another embodiment, the compound of Formula (VI), or its pharmaceutically acceptable salt or prodrug, is provided:

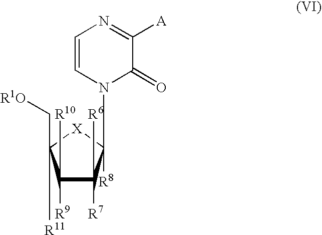

wherein:
wherein A, R¹, R⁶, R⁷, R⁸, R⁹, R¹⁰ and R¹¹ are as defined above.

In one embodiment of any of formulas (I)-(VI), R² and R³ are independently an amino acid. In a subembodiment of any of formulas (I)-(VI), R² and R³ are independently valyl.

In a particularly embodiment, a compound of Formula (VI), or its pharmaceutically acceptable salt or prodrug thereof, is provided in which:
X is O; and/or
each R⁶ is independently an optionally substituted lower alkyl, optionally substituted alkenyl, optionally substituted alkynyl, optionally substituted cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃; and/or
each R⁷ is independently —OH, optionally substituted lower alkyl, optionally substituted alkenyl, optionally substituted alkynyl, optionally substituted cycloalkyl, —O-alkyl, —O-alkenyl, —O-alkynyl, —O-aralkyl, —O-cycloalkyl-, O-acyl, F, Cl, Br, I, CN, NC, SCN, OCN, NCO, NO₂, NH₂, N₃, NH-acyl, NH-alkyl, N-dialkyl, NH-alkenyl, NH-alkynyl, NH-aralkyl, NH-cycloalkyl, SH, S-alkyl, S-alkenyl, S-alkynyl, S-aralkyl, S-acyl, S-cycloalkyl, CO₂-alkyl, CONH-alkyl, CON-dialkyl, CONH-alkenyl, CONH-alkynyl, CONH-aralkyl, CONH-cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH₂, an optionally substituted 3-7 membered carbocyclic, and an optionally substituted 3-7 membered heterocyclic ring having O, S and/or N independently as a heteroatom taken alone or in combination; and/or
each R⁹ is independently hydrogen, optionally substituted lower alkyl, optionally substituted alkenyl, optionally substituted alkynyl, optionally substituted cycloalkyl, —OH, —O-alkyl, —O-alkenyl, —O-alkynyl, —O-aralkyl, —O-cycloalkyl-, O-acyl, F, Cl, Br, I, CN, NC, SCN, OCN, NCO, NO₂, NH₂, N₃, NH-acyl, NH-alkyl, N-dialkyl, NH-alkenyl, NH-alkynyl, NH-aralkyl, NH-cycloalkyl, SH, S-alkyl, S-alkenyl, S-alkynyl, S-aralkyl, S-acyl, S-cycloalkyl, CO₂-alkyl, CONH-alkyl, CON-dialkyl, CONH-alkenyl, CONH-alkynyl, CONH-aralkyl, CONH-cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH₂, an optionally substituted 3-7 membered carbocyclic, and an optionally substituted 3-7 membered heterocyclic ring having O, S and/or N independently as a heteroatom taken alone or in combination; and/or
each R¹⁰ is independently hydrogen, an optionally substituted lower alkyl, optionally substituted alkenyl, optionally substituted alkynyl, optionally substituted cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH; and/or
each R⁸ and R¹¹ is independently H, CH₃, CH₂OH, CH₂F, CH₂N₃, (CH₂)ₘCOOH, (CH₂)ₘCONH₂, and N-acyl; and/or
A is CONH₂; and
each m is independently 1.

### II. METHODS OF USE

In one embodiment, the modified nucleosides of Formula (I)-(VI), and pharmaceutically acceptable salts, esters and prodrugs thereof are provided for use in the treatment or prophylaxis of a flavivirus, pestivirus or hepacivirus infection, especially in individuals diagnosed as having a flavivirus, pestivirus or hepacivirus infection or being at risk for becoming infected by flavivirus, pestivirus or hepacivirus. In one embodiment, a method is provided, comprising administering a treatment effective amount of a compound of Formula (I)-(VI) to a host suffering from or at risk of suffering from a flavivirus, pestivirus or hepacivirus, and in particular HCV, infection. In a particular embodiment, a method of treatment of a host infected with a hepatitis C virus is provided. In another embodiment, the use of a compound of the invention for the treatment of a host infected with a flavivirus, or pestivirus is provided. In a certain embodiment, the virus is not HCV.

The dosages of the compound given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In some embodiments, an anti-hepacivirus, anti-pestivirus or anti-flavivirus compound that exhibits an EC50 of 10-15 μM, or typically less than 1-5 μM, is desirable.

Flaviviruses included within the scope of this invention are discussed generally in Fields Virology, Editors: Fields, B. N., Knipe, D. M., and Howley, P. M., Lippincott-Raven Publishers, Philadelphia, Pa., Chapter 31, 1996. Specific flaviviruses include, without limitation: Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kunlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, Royal Farm, Russian spring-summer encephalitis, Saboya, St. Louis encephalitis, Sal Vieja, San Perlita, Saunarez Reef, Sepik, Sokuluk, Spondweni, Stratford, Tembusu, Tyuleniy, Uganda S, Usutu, Wesselsbron, West Nile, Yaounde, Yellow fever, and Zika.

Pestiviruses included within the scope of this invention are discussed generally in Fields Virology, Editors: Fields, B. N., Knipe, D. M., and Howley, P. M., Lippincott-Raven Publishers, Philadelphia, Pa., Chapter 33, 1996. Specific pestiviruses include, without limitation: bovine viral diarrhea virus (“BVDV”), classical swine fever virus (“CSFV,” also called hog cholera virus), and border disease virus (“BDV”).

The hepacivirus group (hepatitis C virus; HCV) consists of a number of closely related but genotypically distinguishable viruses that infect humans. There are approximately 6 HCV genotypes and more than 50 subtypes. Due to the similarities between pestiviruses and hepaciviruses, combined with the poor ability of hepaciviruses to grow efficiently in cell culture, bovine viral diarrhea virus (BVDV) is often used as a surrogate to study the HCV virus.

The compounds of the invention can be administered via any suitable means. In one embodiment, the compounds of the invention are administered orally. In another embodiment, the compounds are administered parenterally. In yet another embodiment, the compounds are administered via intravenous infusion.

In certain embodiments, the compounds of the invention are administered in a pharmaceutically acceptable carrier or excipient. The carrier or excipient can be useful for the

The modified nucleosides of this invention may inhibit flavivirus, pestivirus or hepacivirus polymerase activity. Nucleosides can be screened for their ability to inhibit flavivirus, pestivirus or hepacivirus polymerase activity in vitro according to screening methods set forth more particularly herein. One can readily determine the spectrum of activity by evaluating the compound in the assays described herein or with another confirmatory assay.

In one embodiment the efficacy of the anti-flavivirus, pestivirus or hepacivirus compound is measured according to the concentration of compound necessary to reduce the plaque number of the virus in vitro, according to methods set forth more particularly herein, by 50% (i.e. the compound's EC₅₀). In a preferred embodiment, the compound exhibits an EC₅₀ of less than 15 or typically, less than 10 micromolar in vitro.

The active compound can be administered as any salt or prodrug that upon administration to the recipient is capable of providing directly or indirectly the parent compound, or that exhibits activity itself. Nonlimiting examples are the pharmaceutically acceptable salts (alternatively referred to as “physiologically acceptable salts”), and a compound, which has been alkylated or acylated at the 5′-position, or on the purine or pyrimidine base (a type of “pharmaceutically acceptable prodrug”). Further, the modifications can affect the biological activity of the compound, in some cases increasing the activity over the parent compound. This can easily be assessed by preparing the salt or prodrug and testing its antiviral activity according to the methods described herein, or other methods known to those skilled in the art.

### III. DEFINITIONS

The term alkyl, as used herein, unless otherwise specified, refers to a saturated straight, branched, or cyclic, primary, secondary, or tertiary hydrocarbon of typically C₁ to C₁₀, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, and 2,3-dimethylbutyl. The term includes both substituted and unsubstituted alkyl groups. Moieties with which the alkyl group can be substituted with one or more substituents selected from the group consisting of halo (F, Cl, Br or I), (e.g. CF₃, 2-Br-ethyl, CH₂F, CH₂Cl, CH₂CF₃ or CF₂CF₃), hydroxyl (e.g. CH₂OH), amino (e.g. CH₂NH₂, CH₂NHCH₃ or CH₂N(CH₃)₂), alkylamino, arylamino, alkoxy, aryloxy, nitro, azido (e.g. CH₂N₃), cyano (e.g. CH₂CN), sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

When a range is referred to in the specification, the range is meant to independently include each and every component of the range. As a non-limiting example of this, when the range C₁₋₆ alkyl is listed, it is meant to independently include C₁-alkyl, C₂-alkyl, C₃-alkyl, C₄-alkyl, C₅-alkyl and C₆-alkyl.

The term lower alkyl, as used herein, and unless otherwise specified, refers to a C₁ to C₄ saturated straight, branched, or if appropriate, a cyclic (for example, cyclopropyl) alkyl group, including both substituted and unsubstituted forms. Unless otherwise specifically stated in this application, when alkyl is a suitable moiety, lower alkyl is typical. Similarly, when alkyl or lower alkyl is a suitable moiety, unsubstituted alkyl or lower alkyl is typical.

The term alkylamino or arylamino refers to an amino group that has one or two alkyl or aryl substituents, respectively.

The term amino acid includes naturally occurring and synthetic α, β γ or δ amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In one embodiment, the amino acid is in the L-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of a natural or synthetic amino acid, including but not limited to α, β γ or δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations.

The term “protected” as used herein and unless otherwise defined refers to a group that is added to an oxygen, nitrogen, or phosphorus atom to prevent its further reaction or for other purposes. A wide variety of oxygen and nitrogen protecting groups are known to those skilled in the art of organic synthesis.

The term aryl, as used herein, and unless otherwise specified, refers to phenyl, biphenyl, or naphthyl, and typically phenyl. The term includes both substituted and unsubstituted moieties. The aryl group can be substituted with one or more moieties selected from the group consisting of hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991.

The term alkaryl or alkylaryl refers to an alkyl group with an aryl substituent. The term aralkyl or arylalkyl refers to an aryl group with an alkyl substituent.

The term halo, as used herein, includes chloro, bromo, iodo, and fluoro.

The term acyl refers to a carboxylic acid ester in which the non-carbonyl moiety of the ester group is selected from straight, branched, or cyclic alkyl or lower alkyl, alkoxyalkyl including methoxymethyl, aralkyl including benzyl, aryloxyalkyl such as phenoxymethyl, aryl including phenyl optionally substituted with halogen, C₁ to C₄ alkyl or C₁ to C₄ alkoxy, sulfonate esters such as alkyl or aralkyl sulphonyl including methanesulfonyl, the mono, di or triphosphate ester, trityl or monomethoxytrityl, substituted benzyl, trialkylsilyl (e.g. dimethyl-t-butylsilyl) or diphenylmethylsilyl. Aryl groups in the esters optimally comprise a phenyl group. The term “lower acyl” refers to an acyl group in which the non-carbonyl moiety is lower alkyl.

As used herein, the term “substantially free of” or “substantially in the absence of” refers to a nucleoside composition that includes at least 85 or 90% by weight, typically 95% to 98% by weight, and even more typically 99% to 100% by weight, of the designated enantiomer of that nucleoside. In one embodiment, in the methods and compounds of this invention, the compounds are substantially free of enantiomers.

Similarly, the term “isolated” refers to a nucleoside composition that includes at least 85 or 90% by weight, typically 95% to 98% by weight, and even more typically 99% to 100% by weight, of the nucleoside, the remainder comprising other chemical species or enantiomers.

The term “independently” is used herein to indicate that the variable, which is independently applied, varies independently from application to application. Thus, in a compound such as R″XYR″, wherein R″ is “independently carbon or nitrogen,” both R″ can be carbon, both R″ can be nitrogen, or one R″ can be carbon and the other R″ nitrogen.

The term host, as used herein, refers to a unicellular or multicellular organism in which the virus can replicate, including cell lines and animals, and typically a human. Alternatively, the host can be carrying a part of the flavivirus, pestivirus or hepacivirus genome, whose replication or function can be altered by the compounds of the present invention. The term host specifically refers to infected cells, cells transfected with all or part of the flavivirus, pestivirus or hepacivirus genome and animals, in particular, primates (including chimpanzees) and humans. In most animal applications of the present invention, the host is a human patient. Veterinary applications, in certain indications, however, are clearly anticipated by the present invention (such as chimpanzees).

The term “pharmaceutically acceptable salt or prodrug” is used throughout the specification to describe any pharmaceutically acceptable form (such as an ester, phosphate ester, salt of an ester or a related group) of a nucleoside compound, which, upon administration to a patient, provides the nucleoside compound. Pharmaceutically acceptable salts include those derived from pharmaceutically acceptable inorganic or organic bases and acids. Suitable salts include those derived from alkali metals such as potassium and sodium, alkaline earth metals such as calcium and magnesium, among numerous other acids well known in the pharmaceutical art. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess antiviral activity against flavivirus, pestivirus or hepacivirus, or are metabolized to a compound that exhibits such activity.

### IV. NUCLEOTIDE SALT OR PRODRUG FORMULATIONS

In cases where compounds are sufficiently basic or acidic to form stable nontoxic acid or base salts, administration of the compound as a pharmaceutically acceptable salt may be appropriate. Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, n ketoglutarate, and □-glycerophosphate. Suitable inorganic salts may also be formed, including, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Any of the nucleosides described herein can be administered as a nucleotide prodrug to increase the activity, bioavailability, stability or otherwise alter the properties of the nucleoside. A nunber of nucleotide prodrug ligands are known. In general, alkylation, acylation or other lipophilic modification of the mono, di or triphosphate of the nucleoside will increase the stability of the nucleotide. Examples of substituent groups that can replace one or more hydrogens on the phosphate moiety are alkyl, aryl, steroids, carbohydrates, including sugars, 1,2-diacylglycerol and alcohols. Many are described in R. Jones and N. Bischofberger, Antiviral Research, 27 (1995) 1-17. Any of these can be used in combination with the disclosed nucleosides to achieve a desired effect.

The active nucleoside can also be provided as a 5′-phosphoether lipid or a 5′-ether lipid, as disclosed in the following references, which are incorporated by reference herein: Kucera, L. S., N. Iyer, E. Leake, A. Raben, Modest E. K., D. L. W., and C. Piantadosi, “Novel membrane-interactive ether lipid analogs that inhibit infectious HIV-1 production and induce defective virus formation,” AIDS Res. Hum. Retro Viruses, 1990, 6, 491-501; Piantadosi, C., J. Marasco C. J., S. L. Morris-Natschke, K. L. Meyer, F. Gumus, J. R. Surles, K. S. Ishaq, L. S. Kucera, N. Iyer, C. A. Wallen, S. Piantadosi, and E. J. Modest, “Synthesis and evaluation of novel ether lipid nucleoside conjugates for anti-HIV activity,” J. Med. Chem., 1991, 34, 1408-1414; Hosteller, K. Y., D. D. Richman, D. A. Carson, L. M. Stuhmiller, G. M. T. van Wijk, and H. van den Bosch, “Greatly enhanced inhibition of human immunodeficiency virus type 1 replication in CEM and HT4-6C cells by 3′-deoxythymidine diphosphate dimyristoylglycerol, a lipid prodrug of 3-deoxythymidine,” Antimicrob. Agents Chemother., 1992, 36, 2025-2029; Hosetler, K. Y., L. M. Stuhmiller, H. B. Lenting, H. van den Bosch, and D. D. Richman, “Synthesis and antiretroviral activity of phospholipid analogs of azidothymidine and other antiviral nucleosides.” J. Biol. Chem., 1990, 265, 61127.

Nonlimiting examples of U.S. patents that disclose suitable lipophilic substituents that can be covalently incorporated into the nucleoside, typically at the 5′-OH position of the nucleoside or lipophilic preparations, include U.S. Pat. Nos. 5,149,794 (Sep. 22, 1992, Yatvin et al.); 5,194,654 (Mar. 16, 1993, Hostetler et al., 5,223,263 (Jun. 29, 1993, Hostetler et al.); 5,256,641 (Oct. 26, 1993, Yatvin et al.); 5,411,947 (May 2, 1995, Hostetler et al.); 5,463,092 (Oct. 31, 1995, Hostetler et al.); 5,543,389 (Aug. 6, 1996, Yatvin et al.); 5,543,390 (Aug. 6, 1996, Yatvin et al.); 5,543,391 (Aug. 6, 1996, Yatvin et al.); and 5,554,728 (Sep. 10, 1996; Basava et al.), all of which are incorporated herein by reference. Foreign patent applications that disclose lipophilic substituents that can be attached to the nucleosides of the present invention, or lipophilic preparations, include WO 89/02733, WO 90/00555, WO 91/16920, WO 91/18914, WO 93/00910, WO 94/26273, WO 96/15132, EP 0 350 287, EP 93917054.4, and WO 91/19721.

### V. COMBINATION AND ALTERNATION THERAPY

It has been recognized that drug-resistant variants of HCV can emerge after prolonged treatment with an antiviral agent. Drug resistance most typically occurs by mutation of a gene that encodes for an enzyme used in viral replication. The efficacy of a drug against HCV infection can be prolonged, augmented, or restored by administering the compound in combination or alternation with a second, and perhaps third, antiviral compound that induces a different mutation from that caused by the principle drug. Alternatively, the pharmacokinetics, biodistriution or other parameter of the drug can be altered by such combination or alternation therapy. In general, combination therapy is typical over alternation therapy because it induces multiple simultaneous stresses on the virus.

Any of the active compounds described herein can be used in combination or alternation with another antiviral compound.

Nonlimiting examples include:

### (1) Interferon

Interferons (IFNs) are compounds that have been commercially available for the treatment of chronic hepatitis for nearly a decade. IFNs are glycoproteins produced by immune cells in response to viral infection. IFNs inhibit viral replication of many viruses, including HCV, and when used as the sole treatment for hepatitis C infection, IFN suppresses serum HCV-RNA to undetectable levels. Additionally, IFN normalizes serum amino transferase levels. Unfortunately, the effects of IFN are temporary and a sustained response occurs in only 8%-9% of patients chronically infected with HCV (Gary L. Davis. Gastroenterology 118:S104-S114, 2000).

A number of patents disclose HCV treatments using interferon-based therapies. For example, U.S. Pat. No. 5,980,884 to Blatt et al. discloses methods for re-treatment of patients afflicted with HCV using consensus interferon. U.S. Pat. No. 5,942,223 to Bazer et al. discloses an anti-HCV therapy using ovine or bovine interferon-tau. U.S. Pat. No. 5,928,636 to Alber et al. discloses the combination therapy of interleukin-12 and interferon alpha for the treatment of infectious diseases including HCV. U.S. Pat. No. 5,908,621 to Glue et al. discloses the use of polyethylene glycol modified interferon for the treatment of HCV. U.S. Pat. No. 5,849,696 to Chretien et al. discloses the use of thymosins, alone or in combination with interferon, for treating HCV. U.S. Pat. No. 5,830,455 to Valtuena et al. discloses a combination HCV therapy employing interferon and a free radical scavenger. U.S. Pat. No. 5,738,845 to Imakawa discloses the use of human interferon tau proteins for treating HCV. Other interferon-based treatments for HCV are disclosed in U.S. Pat. No. 5,676,942 to Testa et al., U.S. Pat. No. 5,372,808 to Blatt et al., and U.S. Pat. No. 5,849,696.

(2) Ribavirin (Battaglia, A. M. et al., Ann. Pharmacother, 2000, 34, 487-494); Berenguer, M. et al. Antivir. Ther., 1998, 3 (Suppl. 3), 125-136).

Ribavirin (1-□D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p 1304, 1989); Rebetol (Schering Plough) and Co-Pegasus (Roche). U.S. Pat. No. 3,798,209 and RE29,835 (ICN Pharmaceuticals) disclose and claim ribavirin. Ribavirin is structurally similar to guanosine, and has in vitro activity against several DNA and RNA viruses including Flaviviridae (Gary L. Davis. Gastroenterology 118:S104-S114, 2000). U.S. Pat. No. 4,211,771 (to ICN Pharmaceuticals) discloses the use of ribavirin as an antiviral agent. Ribavirin reduces serum amino transferase levels to normal in 40% of patients, but it does not lower serum levels of HCV-RNA (Gary L. Davis. Gastroenterology 118:S104-S114, 2000). Thus, ribavirin alone is not effective in reducing viral RNA levels. Additionally, ribavirin has significant toxicity and is known to induce anemia.

### Combination of Interferon and Ribavirin

Schering-Plough sells ribavirin as Rebetol® capsules (200 mg) for administration to patients with HCV. The U.S. FDA has approved Rebetol capsules to treat chronic HCV infection in combination with Schering's alpha interferon-2b products Intron® A and PEG-Intron™. Rebetol capsules are not approved for monotherapy (i.e., administration independent of Intron®A or PEG-Intron), although Intron A and PEG-Intron are approved for monotherapy (i.e., administration without ribavirin). Hoffman La Roche is selling ribavirin under the name Co-Pegasus in Europe and the United States, also for use in combination with interferon for the treatment of HCV. Other alpha interferon products include Roferon-A (Hoffmann-La Roche), Infergen® (Intermune, formerly Amgen's product), and Wellferon® (Wellcome Foundation) are currently FDA-approved for HCV monotherapy. Interferon products currently in development for HCV include: Roferon-A (interferon alfa-2a) by Roche, PEGASYS (pegylated interferon alfa-2a) by Roche, INFERGEN (interferon alfacon-1) by InterMune, OMNIFERON (natural interferon) by Viragen, ALBUFERON by Human Genome Sciences, REBIF (interferon beta-1a) by Ares-Serono, Omega Interferon by BioMedicine, Oral Interferon Alpha by Amarillo Biosciences, and Interferon gamma-1b by InterMune.

The combination of IFN and ribavirin for the treatment of HCV infection has been reported to be effective in the treatment of IFN naïve patients (for example, Battaglia, A. M. et al., Ann. Pharmacother. 34:487-494, 2000). Combination treatment is effective both before hepatitis develops and when histological disease is present (for example, Berenguer, M. et al. Antivir. Ther. 3(Suppl. 3):125-136, 1998). Currently, the most effective therapy for HCV is combination therapy of pegylated interferon with ribavirin (2002 NIH Consensus Development Conference on the Management of Hepatitis C). However, the side effects of combination therapy can be significant and include hemolysis, flu-like symptoms, anemia, and fatigue (Gary L. Davis. Gastroenterology 118:S104-S114, 2000).

(3) Protease inhibitors have been developed for the treatment of Flaviviridae infections. Examples, include, but are not limited to the following

Substrate-based NS3 protease inhibitors (see, for example, Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, particularly hepatitis C virus NS3 protease, PCT WO 98/17679), including alphaketoamides and hydrazinoureas, and inhibitors that terminate in an electrophile such as a boronic acid or phosphonate (see, for example, Llinas-Brunet et al, Hepatitis C inhibitor peptide analogues, PCT WO 99/07734);

Non-substrate-based inhibitors such as 2,4,6-trihydroxy-3-nitro-benzamide derivatives (see, for example, Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group;

Phenanthrenequinones possessing activity against protease, for example in a SDS-PAGE and/or autoradiography assay, such as, for example, Sch 68631, isolated from the fermentation culture broth of Streptomyces sp., (see, for example, Chu M. et al., Tetrahedron Letters, 1996, 37, 7229-7232), and Sch 351633, isolated from the fungus Penicillium griseofulvum, which demonstrates activity in a scintillation proximity assay (see, for example, Chu M. et al., Bioorganic and Medicinal Chemistry Letters 9, 1949-1952); and

Selective NS3 inhibitors, for example, based on the macromolecule elgin c, isolated from leech (see, for example, Qasim M. A. et al., Biochemistry, 1997, 36, 1598-1607). Nanomolar potency against the HCV NS3 protease enzyme has been achieved by the design of selective inhibitors based on the macromolecule eglin c. Eglin c, isolated from leech, is a potent inhibitor of several serine proteases such as S. griseus proteases A and B, □-chymotrypsin, chymase and subtilisin.

Several U.S. patents disclose protease inhibitors for the treatment of HCV. Non-limiting examples include, but are not limited to the following. U.S. Pat. No. 6,004,933 to Spruce et al. discloses a class of cysteine protease inhibitors for inhibiting HCV endopeptidase. U.S. Pat. No. 5,990,276 to Zhang et al. discloses synthetic inhibitors of hepatitis C virus NS3 protease. The inhibitor is a subsequence of a substrate of the NS3 protease or a substrate of the NS4A cofactor. The use of restriction enzymes to treat HCV is disclosed in U.S. Pat. No. 5,538,865 to Reyes et al. Peptides as NS3 serine protease inhibitors of HCV are disclosed in WO 02/008251 to Corvas International, Inc, and WO 02/08187 and WO 02/008256 to Schering Corporation. HCV inhibitor tripeptides are disclosed in U.S. Pat. Nos. 6,534,523, 6,410,531, and 6,420,380 to Boehringer Ingelheim and WO 02/060926 to Bristol Myers Squibb. Diaryl peptides as NS3 serine protease inhibitors of HCV are disclosed in WO 02/48172 to Schering Corporation. Imidazoleidinones as NS3 serine protease inhibitors of HCV are disclosed in WO 02/08198 to Schering Corporation and WO 02/48157 to Bristol Myers Squibb. WO 98/17679 to Vertex Pharmaceuticals and WO 02/48116 to Bristol Myers Squibb also disclose HCV protease inhibitors.

(4) Thiazolidine derivatives, for example, that show relevant inhibition in a reverse-phase HPLC assay with an NS3/4A fusion protein and NS5A/SB substrate (see, for example, Sudo K. et al., Antiviral Research, 1996, 32, 9-18), especially compound RD-1-6250, possessing a fused cinnamoyl moiety substituted with a long alkyl chain, RD4 6205 and RD4 6193;
(5) Thiazolidines and benzanilides, for example, as identified in Kakiuchi N. et al. J. EBS Letters 421, 217-220; Takeshita N. et al. Analytical Biochemistry, 1997, 247, 242-246;
(6) Helicase inhibitors (see, for example, Diana G. D. et al., Compounds, compositions and methods for treatment of hepatitis C, U.S. Pat. No. 5,633,358; Diana G. D. et al., Piperidine derivatives, pharmaceutical compositions thereof and their use in the treatment of hepatitis C, PCT WO 97/36554);
(7) Polymerase inhibitors such as
i) nucleotide analogues, such as gliotoxin (see, for example, Ferrari R. et al. Journal of Virology, 1999, 73, 1649-1654);
ii) the natural product cerulenin (see, for example, Lohmann V. et al., Virology, 1998, 249, 108-118); and
iii) non-nucleoside polymerase inhibitors, including, for example, compound R803 (see, for example, WO 04/018463 A2 and WO 03/040112 A1, both to Rigel Pharmaceuticals, Inc.); substituted diamine pyrimidines (see, for example, WO 03/063794 A2 to Rigel Pharmaceuticals, Inc.); benzimidazole derivatives (see, for example, Bioorg. Med. Chem. Lett., 2004, 14:119-124 and Bioorg. Med. Chem. Lett., 2004, 14:967-971, both to Boehringer Ingelheim Corporation); N,N-disubstituted phenylalanines (see, for example, J. Biol. Chem., 2003, 278:9495-98 and J. Med. Chem., 2003, 13:1283-85, both to Shire Biochem, Inc.); substituted thiophene-2-carboxylic acids (see, for example, Bioorg. Med. Chem. Lett., 2004, 14:793-796 and Bioorg. Med. Chem. Lett., 2004, 14:797-800, both to Shire Biochem, Inc.); □,□-diketoacids (see, for example, J. Med. Chem., 2004, 14-17 and WO 00/006529 A1, both to Merck & Co., Inc.); and meconic acid derivatives (see, for example, Bioorg. Med. Chem. Lett., 2004, 3257-3261, WO 02/006246 A1 and WO03/062211 A1, all to IRBM Merck & Co., Inc.);
   (8) Antisense phosphorothioate oligodeoxynucleotides (S-ODN) complementary, for example, to sequence stretches in the 5′ non-coding region (NCR) of the virus (see, for example, Alt M. et al., Hepatology, 1995, 22, 707-717), or to nucleotides 326-348 comprising the 3′ end of the NCR and nucleotides 371-388 located in the core coding region of the HCV RNA (see, for example, Alt M. et al., Archives of Virology, 1997, 142, 589-599; Galderisi U. et al., Journal of Cellular Physiology, 1999, 181, 251-257).
   (9) Inhibitors of IRES-dependent translation (see, for example, Ikeda N et al., Agent for the prevention and treatment of hepatitis C, Japanese Patent Pub. JP-08268890; Kai Y. et al. Prevention and treatment of viral diseases, Japanese Patent Pub. JP-10101591).
   (10) Nuclease-resistant ribozymes (see, for example, Maccjak, D. J. et al., Hepatology 1999, 30, abstract 995; U.S. Pat. No. 6,043,077 to Barber et al., and U.S. Pat. Nos. 5,869,253 and 5,610,054 to Draper et al.).
   (11) Nucleoside analogs have also been developed for the treatment of Flaviviridae infections.

Idenix Pharmaceuticals, Ltd. discloses branched nucleosides, and their use in the treatment of HCV and flaviviruses and pestiviruses in U.S. Pat. No. 6,914,054, which issued on Jul. 5, 2005, and U.S. Pat. No. 6,812,219, issued Nov. 2, 2004, which correspond to International Publication Nos. WO 01/90121 and WO 01/92282. A method for the treatment of hepatitis C infection (and flaviviruses and pestiviruses) in humans and other host animals is disclosed in the Idenix publications that includes administering an effective amount of a biologically active 1′, 2′, 3′ or 4′-branched GD or CL nucleosides or a pharmaceutically acceptable salt or prodrug thereof, administered either alone or in combination, optionally in a pharmaceutically acceptable carrier. See also U.S. Patent Publication Nos. 2004/0006002 and 2004/0006007 as well as WO 03/026589 and WO 03/026675. Idenix Pharmaceuticals, Ltd. also discloses in US Patent Publication No. 2004/0077587 pharmaceutically acceptable branched nucleoside prodrugs, and their use in the treatment of HCV and flaviviruses and pestiviruses in prodrugs. See also PCT Publication Nos. WO 04/002422, WO 04/002999, WO 04/003000; WO 04/024095 and WO 05/009418.

Biota Inc. discloses various phosphate derivatives of nucleosides, including 1′, 2′, 3′ or 4′-branched □-D or □-L nucleosides, for the treatment of hepatitis C infection in International Patent Publication WO 03/072757.

Emory University and the University of Georgia Research Foundation, Inc. (UGARF) discloses the use of 2′-fluoronucleosides for the treatment of HCV in U.S. Pat. No. 6,348,587. See also US Patent Publication No. 2002/0198171 and International Patent Publication WO 99/43691.

BioChem Pharma Inc. (now Shire Biochem, Inc.) discloses the use of various 1,3-dioxolane nucleosides for the treatment of a Flaviviridae infection in U.S. Pat. No. 6,566,365. See also U.S. Pat. Nos. 6,340,690 and 6,605,614; US Patent Publication Nos. 2002/0099072 and 2003/0225037, as well as International Publication No. WO 01/32153 and WO 00/50424.

BioChem Pharma Inc. (now Shire Biochem, Inc.) also discloses various other 2′-halo, 2′-hydroxy and 2′-alkoxy nucleosides for the treatment of a Flaviviridae infection in US Patent Publication No. 2002/0019363 as well as International Publication No. WO 01/60315 (PCT/CA01/00197; filed Feb. 19, 2001).

ICN Pharmaceuticals, Inc. discloses various nucleoside analogs that are useful in modulating immune response in U.S. Pat. Nos. 6,495,677 and 6,573,248. See also WO 98/16184, WO 01/68663, and WO 02/03997.

U.S. Pat. No. 6,660,721; US Patent Publication Nos. 2003/083307 A1, 2003/008841 A1, and 2004/0110718; as well as International Patent Publication Nos. WO 02/18404; WO 02/100415, WO 02/094289, and WO 04/043159; filed by F. Hoffmann-La Roche AG, discloses various nucleoside analogs for the treatment of HCV RNA replication.

Pharmasset Ltd. discloses various nucleosides and antimetabolites for the treatment of a variety of viruses, including Flaviviridae, and in particular HCV, in US Patent Publication Nos. 2003/0087873, 2004/0067877, 2004/0082574, 2004/0067877, 2004/002479, 2003/0225029, and 2002/00555483, as well as International Patent Publication Nos. WO 02/32920, WO 01/79246, WO 02/48165, WO 03/068162, WO 03/068164 and WO 2004/013298.

Merck & Co., Inc. and Isis Pharmaceuticals disclose in U.S. Pat. No. 6,777,395, issued Aug. 17, 2004; U.S. Patent Publication No. 2004/0072788, 2004/0067901, and 2004/0110717; as well as the corresponding International Patent Publication Nos. WO 02/057425 (PCT/US02/01531; filed Jan. 18, 2002) and WO 02/057287 (PCT/US02/03086; filed Jan. 18, 2002) various nucleosides, and in particular several pyrrolopyrimidine nucleosides, for the treatment of viruses whose replication is dependent upon RNA-dependent RNA polymerase, including Flaviviridae, and in particular HCV. See also WO 2004/000858, WO 2004/003138, WO 2004/007512, and WO 2004/009020.

US Patent Publication No. 2003/028013 A1 as well as International Patent Publication Nos. WO 03/051899, WO 03/061576, WO 03/062255 WO 03/062256, WO 03/062257, and WO 03/061385, filed by Ribapharm, also are directed to the use of certain nucleoside analogs to treat hepatitis C virus.

Genelabs Technologies disclose in US Patent Publication No. 2004/0063658 as well as International Patent Publication Nos. WO 03/093290 and WO 04/028481 various base modified derivatives of nucleosides, including 1′, 2′, 3′ or 4′-branched SD or GL nucleosides, for the treatment of hepatitis C infection.

Eldrup et al. (Oral Session V, Hepatitis C Virus, Flaviviridae; 16^{th} International Conference on Antiviral Research (Apr. 27, 2003, Savannah, Ga.) p. A75) described the structure activity relationship of 2′-modified nucleosides for inhibition of HCV.

Bhat et al (Oral Session V, Hepatitis C Virus, Flaviviridae; 16^{th} International Conference on Antiviral Research (Apr. 27, 2003, Savannah, Ga.); p A75) describe the synthesis and pharmacokinetic properties of nucleoside analogues as possible inhibitors of HCV RNA replication. The authors report that 2′-modified nucleosides demonstrate potent inhibitory activity in cell-based replicon assays.

Olsen et al. (Oral Session V, Hepatitis C Virus, Flaviviridae; 16^{th} International Conference on Antiviral Research (Apr. 27, 2003, Savannah, Ga.) p A76) also described the effects of the 2′-modified nucleosides on HCV RNA replication.

Anti-viral purines that have acyclic substituents are known and have been used to treat various viral infections. Examples of this class of compounds are acyclovir, ganciclovir, famciclovir, penciclovir, adefovir and adefovir dipivoxil, all of which are useful in the treatment of human syncytial virus (HSV), cytomegalo virus (CMV), and varicella-zoster virus (see EP 0 72027 to the Wellcome Foundation Ltd., UK, for treatment of equine rhinopneumonitis virus; JP 06227982 to Ajinomoto KK, for treatment of varicella-zoster virus and cytomegalovirus; S. Vittori et al., Deaza- and Deoxyadenosine Derivatives: Synthesis and Inhibition of Animal Viruses as Human Infection Models, in Nucleosides. Nucleotides & Nucleic Acids (2003) 22(5-8): 877-881, for treatment of bovine herpes virus 1 (BHV-1) and sheep Maedi-Visna Virus (MVV); R. Wang et al., Synthesis and biological activity of 2-aminopurine methylenecyclo-propane analogues of nucleosides, in Nucleosides, Nucleotides & Nucleic Acids (2003) 22(2): 135-144, for treatment of HSV-1 and VZV; U.S. Pat. No. 6,444,656 to BioChem Pharma, Inc., Canada, for treatment of HIV and/or HBV infections; and WO 02/057288 to LG Chem Investment Ltd. for acyclic nucleoside phosphonate compounds for use as anti-HBV agents).

(12) Other miscellaneous compounds including 1-amino-alkylcyclohexanes (for example, U.S. Pat. No. 6,034,134 to Gold et al.), alkyl lipids (for example, U.S. Pat. No. 5,922,757 to Chojkier et al.), vitamin E and other antioxidants (for example, U.S. Pat. No. 5,922,757 to Chojkier et al.), squalene, amantadine, bile acids (for example, U.S. Pat. No. 5,846,964 to Ozeki et al.), N-(phosphonoacetyl)-L-aspartic acid (for example, U.S. Pat. No. 5,830,905 to Diana et al.), benzenedicarboxamides (for example, U.S. Pat. No. 5,633,388 to Diana et al.), polyadenylic acid derivatives (for example, U.S. Pat. No. 5,496,546 to Wang et al.), 2′,3′-dideoxyinosine (for example, U.S. Pat. No. 5,026,687 to Yarchoan et al.), benzimidazoles (for example, U.S. Pat. No. 5,891,874 to Colacino et al.), plant extracts (for example, U.S. Pat. No. 5,837,257 to Tsai et al., U.S. Pat. No. 5,725,859 to Omer et al., and U.S. Pat. No. 6,056,961), and piperidenes (for example, U.S. Pat. No. 5,830,905 to Diana et al.).
(13) Other compounds include, for example: Interleukin-10 by Schering-Plough, IP-501 by Interneuron, Merimebodib VX-497 by Vertex, AMANTADINE® (Symmetrel) by Endo Labs Solvay, HEPTAZYMEW by RPI, IDN-6556 by Idun Pharma., XTL-002 by XTL., HCV/MF59 by Chiron, CIVACIR® (Hepatitis C Immune Globulin) by NABI, LEVOVIRIN® by ICN/Ribapharm, VIRAMIDINE® by ICN/Ribapharm, ZADAXIN® (thymosin alfa-1) by Sci Clone, thymosin plus pegylated interferon by Sci Clone, CEPLENE® (histamine dihydrochloride) by Maxim, VX 950/LY 570310 by Vertex/Eli Lilly, ISIS14803 by Isis Pharmaceutical/Elan, IDN-6556 by Idun Pharmaceuticals, Inc., JTK 003 by AKROS Pharma, BILN-2061 by Boehringer Ingelheim, CellCept (mycophenolate mofetil) by Roche, T67, a □-tubulin inhibitor, by Tularik, a therapeutic vaccine directed to E2 by Innogenetics, FK788 by Fujisawa Healthcare, Inc., IdB 1016 (Siliphos, oral silybin-phosphatdylcholine phytosome), RNA replication inhibitors (VP50406) by ViroPharma/Wyeth, therapeutic vaccine by Intercell, therapeutic vaccine by Epimmune/Genencor, IRES inhibitor by Anadys, ANA 245 and ANA 246 by Anadys, immunotherapy (Therapore) by Avant, protease inhibitor by Corvas/SChering, helicase inhibitor by Vertex, fusion inhibitor by Trimeris, T cell therapy by CellExSys, polymerase inhibitor by Biocryst, targeted RNA chemistry by PTC Therapeutics, Dication by Immtech, Int., protease inhibitor by Agouron, protease inhibitor by Chiron/Medivir, antisense therapy by AVI BioPharma, antisense therapy by Hybridon, hemopurifier by Aethlon Medical, therapeutic vaccine by Merix, protease inhibitor by Bristol-Myers Squibb/Axys, Chron-VacC, a therapeutic vaccine, by Tripep, UT 231B by United Therapeutics, protease, helicase and polymerase inhibitors by Genelabs Technologies, IRES inhibitors by Immusol, R803 by Rigel Pharmaceuticals, INFERGEN® (interferon alphacon-1) by InterMune, OMNIFERON® (natural interferon) by Viragen, ALBUFERON® by Human Genome Sciences, REBIF® (interferon beta-1a) by Ares-Serono, Omega Interferon by BioMedicine, Oral Interferon Alpha by Amarillo Biosciences, interferon gamma, interferon tau, and Interferon gamma-1b by InterMune.

### VI. PHARMACEUTICAL COMPOSITIONS

Host, including humans, infected with flavivirus, pestivirus or hepacivirus can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

Nonlimiting examples of doses of the compound infection will be in the range from 1 to 80 mg/kg, 1 to 70 mg/kg, 1 to 60 mg/kg, 1 to 50 mg/kg, or 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent nucleoside to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3000 mg, typically 70 to 1400 mg of active ingredient per unit dosage form. A oral dosage of 50-1000 mg is usually convenient.

Ideally the active ingredient should be administered to achieve peak plasma concentrations of the active compound of from about 0.2 to 70 μM, typically about 1.0 to 10 μM. This may be achieved, for example, by the intravenous injection of a 0.1 to 5% solution of the active ingredient, optionally in saline, or administered as a bolus of the active ingredient.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

One mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic.

If administered intravenously, typical carriers are physiological saline or phosphate buffered saline (PBS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antibodies to viral antigens) are also typical as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

### VII. PROCESSES FOR THE PREPARATION OF ACTIVE COMPOUNDS

The nucleosides of the present invention can be synthesized by any means known in the art. In particular, the synthesis of the present nucleosides can be achieved by either alkylating the appropriately modified sugar, followed by glycosylation or glycosylation followed by alkylation of the nucleoside. The following non-limiting embodiments illustrate some general methodology to obtain the nucleosides of the present invention.

### General Synthesis of 1′-C-Branched Nucleosides

1′-C-Branched ribonucleosides of the following structure:

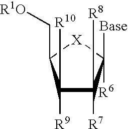

wherein Base, R¹, R², R³, R⁴, R⁵, R⁶, R⁷, R⁸, R⁹, R¹, Y, W, W², W³, X, X¹, X² and X³ are as defined herein can be prepared by one of the following general methods.
1) Modification from the Lactone

The key starting material for this process is an appropriately substituted lactone. The lactone can be purchased or can be prepared by any known means including standard epimerization, substitution and cyclization techniques. The lactone can be optionally protected with a suitable protecting group, typically with an acyl or silyl group, by methods well known to those skilled in the art, as taught by Greene et al. Protective Groups in Organic Synthesis, John Wiley and Sons, Second Edition, 1991. The protected lactone can then be coupled with a suitable coupling agent, such as an organometallic carbon nucleophile, such as a Grignard reagent, an organolithium, lithium dialkylcopper or R⁶—SiMe₃ in TBAF with the appropriate non-protic solvent at a suitable temperature, to give the 1′-alkylated sugar.

The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 1′-C-branched ribonucleoside is desired. The synthesis of a ribonucleoside is shown in Scheme 1. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

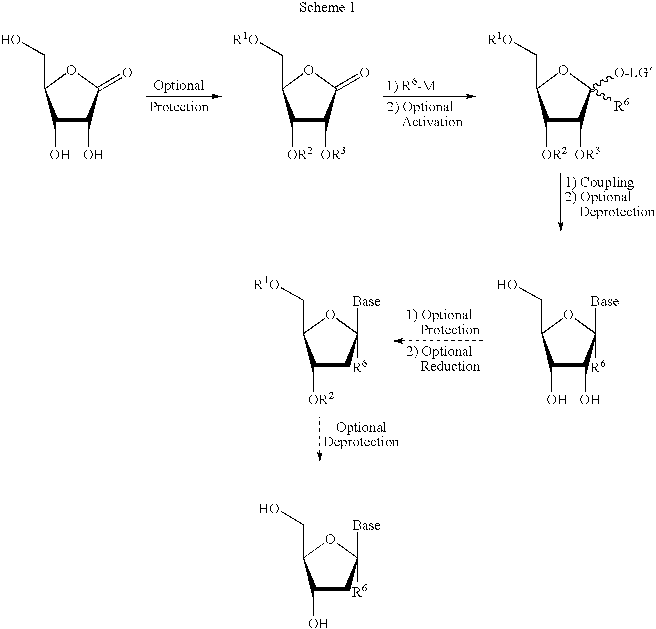

2. Alternative method for the preparation of 1′-C-branched nucleosides

The key starting material for this process is an appropriately substituted hexose. The hexose can be purchased or can be prepared by any known means including standard epimerization (e.g. via alkaline treatment), substitution and coupling techniques. The hexose can be selectively protected to give the appropriate hexa-furanose, as taught by Townsend Chemistry of Nucleosides and Nucleotides, Plenum Press, 1994.

The 1′-hydroxyl can be optionally activated to a suitable leaving group such as an acyl group or a halogen via acylation or halogenation, respectively. The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

The 1′-CH₂—OH, if protected, can be selectively deprotected by methods well known in the art. The resultant primary hydroxyl can be functionalized to yield various C-branched nucleosides. For example, the primary hydroxyl can be reduced to give the methyl, using a suitable reducing agent. Alternatively, the hydroxyl can be activated prior to reduction to facilitate the reaction; i.e. via the Barton reduction. In an alternate embodiment, the primary hydroxyl can be oxidized to the aldehyde, then coupled with a carbon nucleophile, such as a Grignard reagent, an organolithium, lithium dialkylcopper or R⁶—SiMe₃ in TBAF with the appropriate non-protic solvent at a suitable temperature.

In a particular embodiment, the 1′-C-branched ribonucleoside is desired. The synthesis of a ribonucleoside is shown in Scheme 2. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

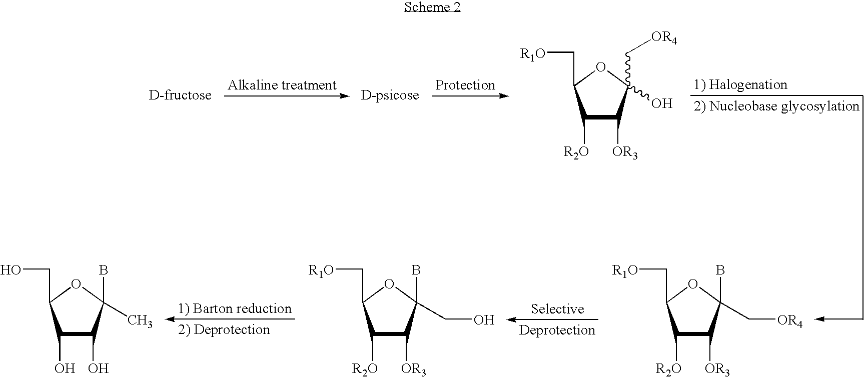

In addition, the L-enantiomers corresponding to the compounds of the invention can be prepared following the same general methods (1 or 2), beginning with the corresponding L-sugar or nucleoside L-enantiomer as starting material.

### General Synthesis of 2′-C-Branched Nucleosides

2′-C-Branched ribonucleosides of the following structure:

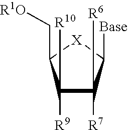

wherein Base, R¹, R², R³, R⁴, R⁵, R⁶, R⁷, R⁹, R¹⁰, Y, W¹, W², W³, X, X¹, X² and X³ are as defined herein can be prepared by one of the following general methods.
1. Glycosylation of the Nucleobase with an Appropriately Modified Sugar

The key starting material for this process is an appropriately substituted sugar with a 2′-OH and 2′-H, with the appropriate leaving group (LG), for example an acyl group or a halogen. The sugar can be purchased or can be prepared by any known means including standard epimerization, substitution, oxidation and reduction techniques. The substituted sugar can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Then coupling of an organometallic carbon nucleophile, such as a Grignard reagent, an organolithium, lithium dialkylcopper or R⁶—SiMe₃ in TBAF with the ketone with the appropriate non-protic solvent at a suitable temperature, yields the 2′-alkylated sugar. The alkylated sugar can be optionally protected with a suitable protecting group, typically with an acyl or silyl group, by methods well known to those skilled in the art, as taught by Greene et al. Protective Groups in Organic Synthesis, John Wiley and Sons, Second Edition, 1991.

The optionally protected sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 2′-C-branched ribonucleoside is desired. The synthesis of a ribonucleoside is shown in Scheme 3. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

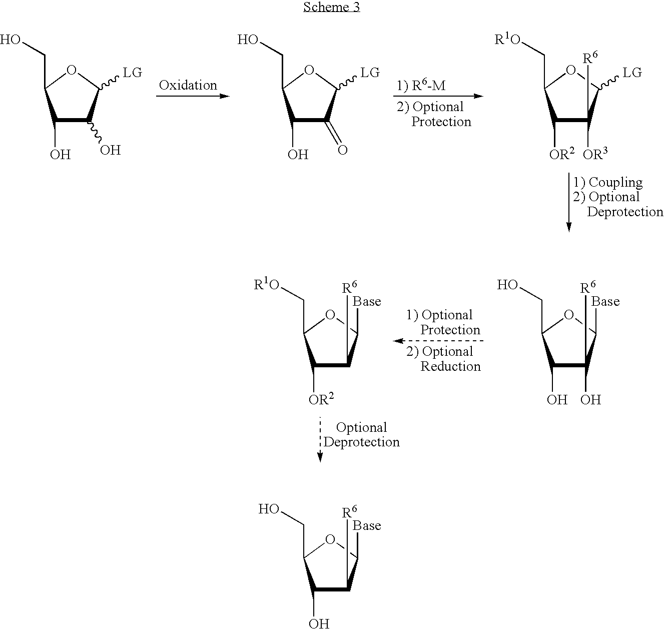

### 2. Modification of a Preformed Nucleoside

The key starting material for this process is an appropriately substituted nucleoside with a 2′-OH and 2′-H. The nucleoside can be purchased or can be prepared by any known means including standard coupling techniques. The nucleoside can be optionally protected with suitable protecting groups, typically with acyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂-CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Subsequently, the nucleoside can be deprotected by methods well known to those skilled in the art, as taught by GreeneGreene et al. Protective Groups in Organic Synthesis, John Wiley and Sons, Second Edition, 1991.

In a particular embodiment, the 2′-C-branched ribonucleoside is desired. The synthesis of a ribonucleoside is shown in Scheme 4. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

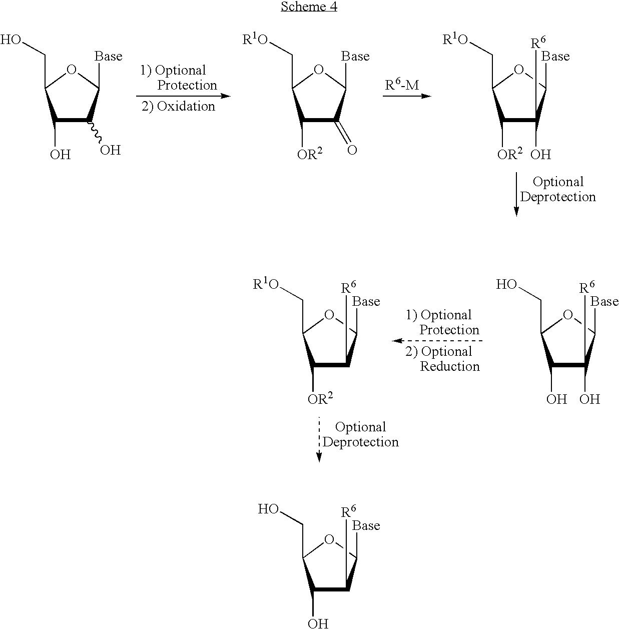

In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

### General Synthesis of 3′-C-Branched Nucleosides

3′-C-Branched ribonucleosides of the following structure:

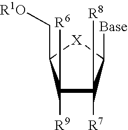

wherein Base, R¹, R², R³, R⁴, R⁵, R⁶, R⁷, R⁸, R⁹, Y, W¹, W², W³, X, X¹, X² and X³ are as defined herein can be prepared by one of the following general methods.
1 Glycosylation of the Nucleobase with an Appropriately Modified Sugar

The key starting material for this process is an appropriately substituted sugar with a 3′-OH and 3′-H, with the appropriate leaving group (LG), for example an acyl group or a halogen. The sugar can be purchased or can be prepared by any known means including standard epimerization, substitution, oxidation and reduction techniques. The substituted sugar can then be oxidized with the appropriate oxidizing agent in a compatible solvent at a suitable temperature to yield the 3′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Then coupling of an organometallic carbon nucleophile, such as a Grignard reagent, an organolithium, lithium dialkylcopper or R⁶—SiMe₃ in TBAF with the ketone with the appropriate non-protic solvent at a suitable temperature, yields the 3′-C-branched sugar. The 3′-C-branched sugar can be optionally protected with a suitable protecting group, typically with an acyl or silyl group, by methods well known to those skilled in the art, as taught by Greene et al. Protective Groups in Organic Synthesis, John Wiley and Sons, Second Edition, 1991.

The optionally protected sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature. Alternatively, a halo-sugar can be coupled to a silylated base with the presence of trimethylsilyltriflate.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. The synthesis of a ribonucleoside is shown in Scheme 5. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

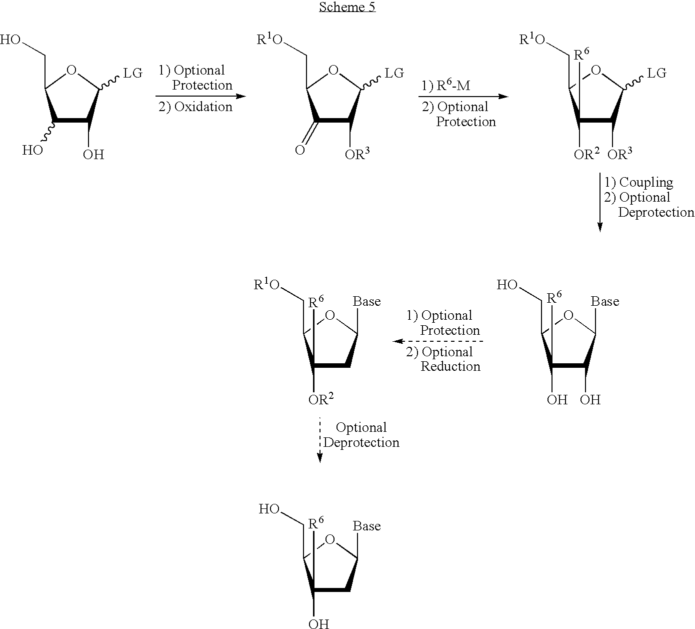

### 2. Modification of a Pre-Formed Nucleoside

The key starting material for this process is an appropriately substituted nucleoside with a 3′-OH and 3′-H. The nucleoside can be purchased or can be prepared by any known means including standard coupling techniques. The nucleoside can be optionally protected with suitable protecting groups, typically with acyl or silyl groups, by methods well known to those skilled in the art, as taught by Greene et al. Protective Groups in Organic Synthesis, John Wiley and Sons, Second Edition, 1991.

The appropriately protected nucleoside can then be oxidized with the appropriate oxidizing agent in a compatible solvent at a suitable temperature to yield the 2′-modified sugar. Possible oxidizing agents are Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₁₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 3′-C-branched ribonucleoside is desired. The synthesis of a ribonucleoside is shown in Scheme 6. Alternatively, deoxyribo-nucleoside is desired. To obtain these nucleosides, the formed ribonucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

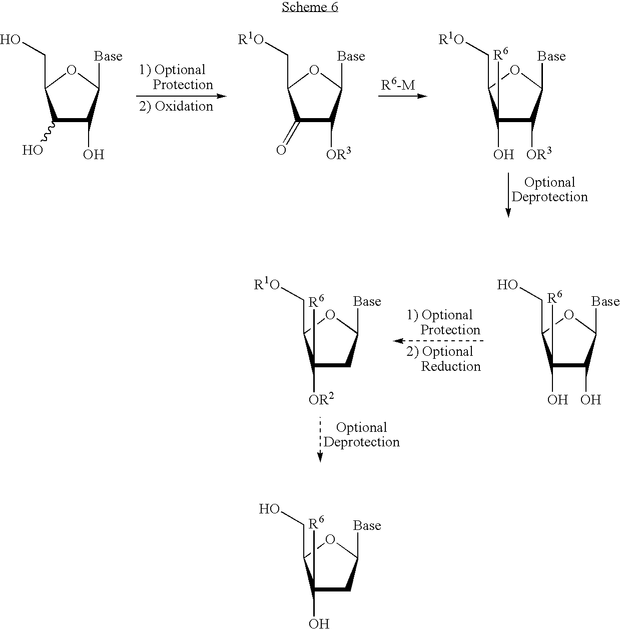

In another embodiment of the invention, the L-enantiomers are desired. Therefore, the L-enantiomers can be corresponding to the compounds of the invention can be prepared following the same foregoing general methods, beginning with the corresponding L-sugar or nucleoside L-enantiomer as starting material.

### General Synthesis of 4′-C-Branched Nucleosides

4′-C-Branched ribonucleosides of the following structure:

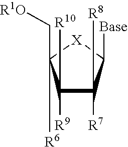

wherein Base, R¹, R², R³, R⁴, R⁵, R⁶, R⁷, R⁸, R⁹, R¹⁰, Y, W¹, W², W³, X, X¹, X² and X¹ are as defined herein can be prepared by one of the following general methods.
1. Modification from the Pentodialdo-Furanose

The key starting material for this process is an appropriately substituted pentodialdo-furanose. The pentodialdo-furanose can be purchased or can be prepared by any known means including standard epimerization, substitution and cyclization techniques.

In one embodiment, the pentodialdo-furanose is prepared from the appropriately substituted hexose. The hexose can be purchased or can be prepared by any known means including standard epimerization (e.g. via alkaline treatment), substitution and coupling techniques. The hexose can be either in the furanose form, or cyclized via any means known in the art, such as methodology taught by Townsend Chemistry of Nucleosides and Nucleotides, Plenum Press, 1994, typically by selectively protecting the hexose, to give the appropriate hexafuranose.

The 4′-hydroxymethylene of the hexafuranose then can be oxidized with the appropriate oxidizing agent in a compatible solvent at a suitable temperature to yield the 4′-aldo-modified sugar. Possible oxidizing agents are Swern reagents, Jones reagent (a mixture of chromic acid and sulfuric acid), Collins's reagent (dipyridine Cr(VI) oxide, Corey's reagent (pyridinium chlorochromate), pyridinium dichromate, acid dichromate, potassium permanganate, MnO₂, ruthenium tetroxide, phase transfer catalysts such as chromic acid or permanganate supported on a polymer, Cl₁₂-pyridine, H₂O₂-ammonium molybdate, NaBrO₂—CAN, NaOCl in HOAc, copper chromite, copper oxide, Raney nickel, palladium acetate, Meerwin-Pondorf-Verley reagent (aluminum t-butoxide with another ketone) and N-bromosuccinimide, though typically using H₃PO₄, DMSO and DCC in a mixture of benzene/pyridine at room temperature.

Then, the pentodialdo-furanose can be optionally protected with a suitable protecting group, typically with an acyl or silyl group, by methods well known to those skilled in the art, as taught by Greene et al. Protective Groups in Organic Synthesis, John Wiley and Sons, Second Edition, 1991. In the presence of a base, such as sodium hydroxide, the protected pentodialdo-furanose can then be coupled with a suitable electrophilic alkyl, halogeno-alkyl (i.e. CF₃), alkenyl or alkynyl (i.e. allyl), to obtain the 4′-alkylated sugar. Alternatively, the protected pentodialdo-furanose can be coupled with the corresponding carbonyl, such as formaldehyde, in the presence of a base, such as sodium hydroxide, with the appropriate polar solvent, such as dioxane, at a suitable temperature, which can then be reduced with an appropriate reducing agent to give the 4′-alkylated sugar. In one embodiment, the reduction is carried out using PhOC(S)CI, DMAP, typically in acetonitrile at room temperature, followed by treatment of ACCN and TMSS refluxed in toluene.

The optionally activated sugar can then be coupled to the BASE by methods well known to those skilled in the art, as taught by Townsend Chemistry of Nucleosides and Nucleotides, Plenum Press, 1994. For example, an acylated sugar can be coupled to a silylated base with a lewis acid, such as tin tetrachloride, titanium tetrachloride or trimethylsilyltriflate in the appropriate solvent at a suitable temperature.

Subsequently, the nucleoside can be deprotected by methods well known to those skilled in the art, as taught by Greene et al. Protective Groups in Organic Synthesis, John Wiley and Sons, Second Edition, 1991.

In a particular embodiment, the 4′-C-branched ribonucleoside is desired. Alternatively, deoxyribonucleoside is desired. To obtain these deoxyribo-nucleosides, a formed ribo-nucleoside can optionally be protected by methods well known to those skilled in the art, as taught by Greene et al. Protective Groups in Organic Synthesis, John Wiley and Sons, Second Edition, 1991, and then the 2′-OH can be reduced with a suitable reducing agent. Optionally, the 2′-hydroxyl can be activated to facilitate reduction; i.e. via the Barton reduction.

In another embodiment of the invention, the L-enantiomers are desired. Therefore, the L-enantiomers can be corresponding to the compounds of the invention can be prepared following the same foregoing general methods, beginning with the corresponding L-pentodialdo-furanose as starting material.

The following table shows a number of compounds that were prepared using the procedures described above:

| Structure | Formula | Weight |
|---|---|---|
| 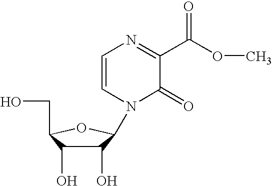 | C11 H14 N2 O7 | 286.2386 |
| 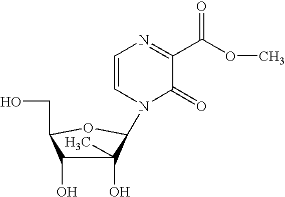 | C12 H16 N2 O7 | 300.2654 |
| 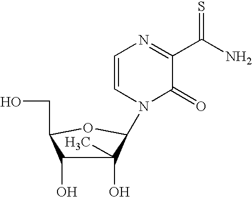 | C11 H15 N3 O5 S | 301.3215 |
| 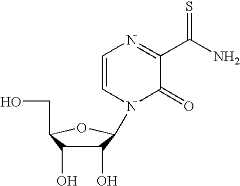 | C10 H13 N3 O5 S | 287.2947 |
| 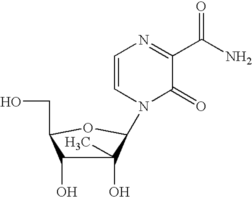 | C11 H15 N3 O6 | 285.2545 |
| 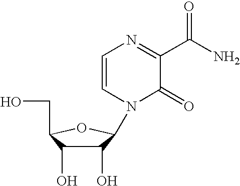 | C10 H13 N3 O6 | 271.2277 |
| 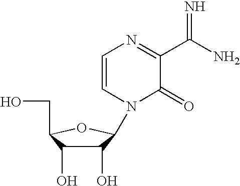 | C10 H14 N4 O5 | 270.2436 |
| 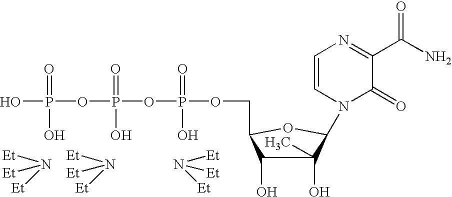 | C11 H18 N3 O15 P3 . 3 C6 H15 N | 828.7657 |
| 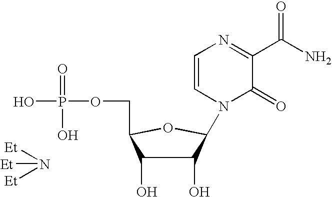 | C10 H14 N3 O9 P . C6 H15 N | 452.3981 |
| 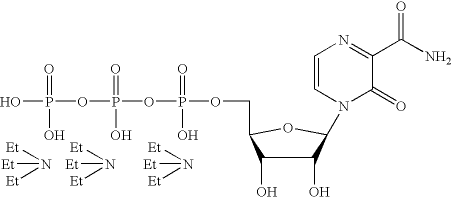 | C10 H16 N3 O15 P3 . 3 C6 H15 N | 814.7389 |
| 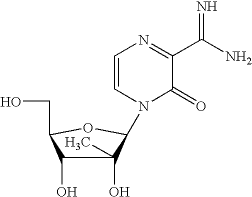 | C11 H16 N4 O5 | 284.2704 |
| 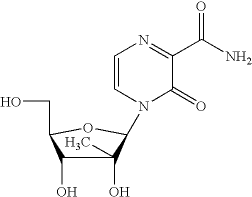 | C11 H15 N3 O6 | 285.2545 |
| 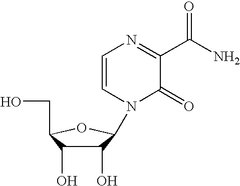 | C10 H13 N3 O6 | 271.2277 |

The present invention is described by way of illustration, in the following examples. It will be understood by one of ordinary skill in the art that these examples are in no way limiting and that variations of detail can be made without departing from the spirit and scope of the present invention.

### VIII. ANTI-FLAVIVIRUS, PESTIVIRUS OR HEPACIVIRUS ACTIVITY

Compounds can exhibit anti-flavivirus, pestivirus or hepacivirus activity by inhibiting flavivirus, pestivirus or hepacivirus polymerase, by inhibiting other enzymes needed in the replication cycle, or by other pathways.

### EXAMPLE 1

### Phosphorylation Assay of Nucleoside to Active Triphosphate

To determine the cellular metabolism of the compounds, HepG2 cells are obtained from the American Type Culture Collection (Rockville, Md.), and are grown in 225 cm² tissue culture flasks in minimal essential medium supplemented with non-essential amino acids, 1% penicillin-streptomycin. The medium is renewed every three days, and the cells are subcultured once a week. After detachment of the adherent monolayer with a 10 minute exposure to 30 mL of trypsin-EDTA and three consecutive washes with medium, confluent HepG2 cells are seeded at a density of 2.5×10⁶ cells per well in a 6-well plate and exposed to 10 μM of [³H] labeled active compound (500 dpm/pmol) for the specified time periods. The cells are maintained at 37° C. under a 5% CO₂ atmosphere. At the selected time points, the cells are washed three times with ice-cold phosphate-buffered saline (PBS). Intracellular active compound and its respective metabolites are extracted by incubating the cell pellet overnight at −20° C. with 60% methanol followed by extraction with an additional 20 μL of cold methanol for one hour in an ice bath. The extracts are then combined, dried under gentle filtered air flow and stored at −20° C. until HPLC analysis.

### EXAMPLE 2

### Bioavailability Assay in Cynomolgus Monkeys

Within 1 week prior to the study initiation, the cynomolgus monkey is surgically implanted with a chronic venous catheter and subcutaneous venous access port (VAP) to facilitate blood collection and undergoes a physical examination including hematology and serum chemistry evaluations and the body weight is recorded. Each monkey (six total) receives approximately 250 μCi of ³H activity with each dose of active compound at a dose level of 10 mg/kg at a dose concentration of 5 mg/mL, either via an intravenous bolus (3 monkeys, IV), or via oral gavage (3 monkeys, PO). Each dosing syringe is weighed before dosing to gravimetrically determine the quantity of formulation administered. Urine samples are collected via pan catch at the designated intervals (approximately 18-0 hours pre-dose, 0-4, 4-8 and 8-12 hours post-dosage) and processed. Blood samples are collected as well (pre-dose, 0.25, 0.5, 1, 2, 3, 6, 8, 12 and 24 hours post-dosage) via the chronic venous catheter and VAP or from a peripheral vessel if the chronic venous catheter procedure should not be possible. The blood and urine samples are analyzed for the maximum concentration (Cₘₐₓ), time when the maximum concentration is achieved (Tₘₐₓ), area under the curve (AUC), half life of the dosage concentration (T_{1/2}), clearance (CL), steady state volume and distribution (Vₛₛ) and bioavailability (F).

### EXAMPLE 3

### Bone Marrow Toxicity Assay

Human bone marrow cells are collected from normal healthy volunteers and the mononuclear population are separated by Ficoll-Hypaque gradient centrifugation as described previously by Sommadossi J-P, Carlisle R. “Toxicity of 3′-azido-3′-deoxythymidine and 9-(1,3-dihydroxy-2-propoxymethyl)guanine for normal human hematopoietic progenitor cells in vitro” Antimicrobial Agents and Chemotherapy 1987; 31:452-454; and Sommadossi J-P, Schinazi R F, Chu C K, Xie M-Y. “Comparison of cytotoxicity of the (−)- and (+)-enantiomer of 2′,3′-dideoxy-3′-thiacytidine in normal human bone marrow progenitor cells” Biochemical Pharmacology 1992; 44:1921-1925. The culture assays for CFU-GM and BFU-E are performed using a bilayer soft agar or methylcellulose method. Drugs are diluted in tissue culture medium and filtered. After 14 to 18 days at 37° C. in a humidified atmosphere of 5% CO₂ in air, colonies of greater than 50 cells are counted using an inverted microscope. The results are presented as the percent inhibition of colony formation in the presence of drug compared to solvent control cultures.

### EXAMPLE 4

### Mitochondria Toxicity Assay

HepG2 cells are cultured in 12-well plates as described above and exposed to various concentrations of drugs as taught by Pan-Zhou X-R, Cui L, Zhou X-J, Sommadossi J-P, Darley-Usmer VM. “Differential effects of antiretroviral nucleoside analogs on mitochondrial function in HepG2 cells” Antimicrob Agents Chemother 2000; 44:496-503. Lactic acid levels in the culture medium after 4 day drug exposure are measured using a Boehringer lactic acid assay kit. Lactic acid levels are normalized by cell number as measured by hemocytometer count.

### EXAMPLE 5

### Cytotoxicity Assay

Cells are seeded at a rate of between 5×10³ and 5×10⁴/well into 96-well plates in growth medium overnight at 37° C. in a humidified CO₂ (5%) atmosphere. New growth medium containing serial dilutions of the drugs is then added. After incubation for 4 days, cultures are fixed in 50% TCA and stained with sulforhodamine B. The optical density is read at 550 nm. The cytotoxic concentration is expressed as the concentration required to reduce the cell number by 50% (CC₅₀).

### EXAMPLE 6

### Cell Protection Assay (CPA)

The assay is performed essentially as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986. MDBK cells (ATCC) are seeded onto 96-well culture plates (4,000 cells per well) 24 hours before use. After infection with BVDV (strain NADL, ATCC) at a multiplicity of infection (MOI) of 0.02 plaque forming units (PFU) per cell, serial dilutions of test compounds are added to both infected and uninfected cells in a final concentration of 0.5% DMSO in growth medium. Each dilution is tested in quadruplicate. Cell densities and virus inocula are adjusted to ensure continuous cell growth throughout the experiment and to achieve more than 90% virus-induced cell destruction in the untreated controls after four days post-infection. After four days, plates are fixed with 50% TCA and stained with sulforhodamine B. The optical density of the wells is read in a microplate reader at 550 nm. The 50% effective concentration (EC₅₀) values are defined as the compound concentration that achieved 50% reduction of cytopathic effect of the virus.

### Plaque Reduction Assay

For each compound the effective concentration is determined in duplicate 24-well plates by plaque reduction assays. Cell monolayers are infected with 100 PFU/well of virus. Then, serial dilutions of test compounds in MEM supplemented with 2% inactivated serum and 0.75% of methyl cellulose are added to the monolayers. Cultures are further incubated at 37° C. for 3 days, then fixed with 50% ethanol and 0.8% Crystal Violet, washed and air-dried. Then plaques are counted to determine the concentration to obtain 90% virus suppression.

### EXAMPLE 7

### Yield Reduction Assay

For each compound the concentration to obtain a 6-log reduction in viral load is determined in duplicate 24-well plates by yield reduction assays. The assay is performed as described by Baginski, S. G.; Pevear, D. C.; Seipel, M.; Sun, S. C. C.; Benetatos, C. A.; Chunduru, S. K.; Rice, C. M. and M. S. Collett “Mechanism of action of a pestivirus antiviral compound” PNAS USA 2000, 97(14), 7981-7986, with minor modifications. Briefly, MDBK cells are seeded onto 24-well plates (2×105 cells per well) 24 hours before infection with BVDV (NADL strain) at a multiplicity of infection (MOI) of 0.1 PFU per cell. Serial dilutions of test compounds are added to cells in a final concentration of 0.5% DMSO in growth medium. Each dilution is tested in triplicate. After three days, cell cultures (cell monolayers and supernatants) are lysed by three freeze-thaw cycles, and virus yield is quantified by plaque assay. Briefly, MDBK cells are seeded onto 6-well plates (5×105 cells per well) 24 h before use. Cells are inoculated with 0.2 mL of test lysates for 1 hour, washed and overlaid with 0.5% agarose in growth medium. After 3 days, cell monolayers are fixed with 3.5% formaldehyde and stained with 1% crystal violet (w/v in 50% ethanol) to visualize plaques. The plaques are counted to determine the concentration to obtain a 6-log reduction in viral load.

This invention has been described with reference to certain embodiments. Variations and modifications of the invention, will be obvious to those skilled in the art from the foregoing detailed description of the invention.

We claim:

1. A compound of Formula (I) or (II): 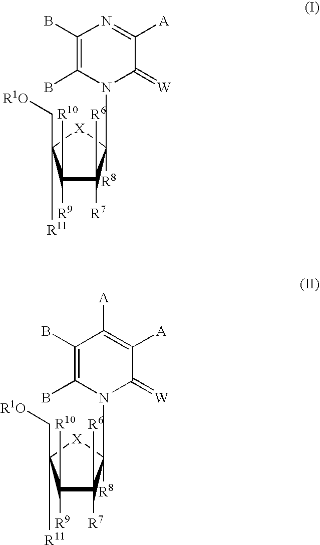
or a pharmaceutically acceptable salt or ester thereof, wherein:
R¹ is independently H, optionally substituted alkyl; acyl; phosphate; sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹ is independently H or phosphate;
each A is independently a straight, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, SH, —S-alkyl, —S-acyl, —S-aryl, —S-cycloalkyl, —S-aralkyl, —CO₂-alkyl, —CONH-alkyl, —CON-dialkyl, CF₃, —CHₘOH, —(CH₂)ₘNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each B is independently H, a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, —CO—O-alkoxyalkyl, —CONHR⁴, —C(NR⁴)N(R⁴)₂, —C(S)N(R⁴)₂, —CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, SH, —S-alkyl, —S-acyl, —S-aryl, —S-cycloalkyl, —S-aralkyl, —CO₂-alkyl, —CONH-alkyl, —CON-dialkyl, CF₃, —CHₘOH, —(CH₂)ₘNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each Y³ is independently H, F, Cl, Br or I;
each R⁴ and R⁵ is independently hydrogen, acyl, alkyl, lower alkyl, alkenyl, alkynyl or cycloalkyl.
X is O or CH;
each R⁶ is independently an optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, —(CH₂)ₘC(O)N(R⁴)₂, C(O)OR⁴ or cyano;
each R⁷ is independently OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle, optionally substituted heterocycle, optionally substituted heteroaryl, —(CH₂)ₘC(O)OR⁴, (CH₂)ₘC(6)SR⁴—(CH₂)ₘC(O)NHR⁴, —(CH₂)ₙC(O)N(R⁴)₂, —C(O)OR⁴, —C(O)SR⁴, —O(R⁴), S(R⁴), NO₂, —NR⁴R⁵, azido, cyano, SCN, OCN, NCO or halo;
each R⁸ and R¹¹ is independently hydrogen, an optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, haloalkenyl, Br-vinyl, optionally substituted alkynyl, haloalkynyl, —CH₂C(O)N(R⁴)₂, —(CH₂)ₘC(O)OR¹, —(CH₂)ₘC(O)NHR⁴, —C(O)OR⁴, cyano, NH-acyl or N(acyl)₂;
each R⁹ and R¹⁰ are independently hydrogen, OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, NO₂, haloalkenyl, Br-vinyl, optionally substituted alkynyl, haloalkynyl, optionally substituted carbocycle, optionally substituted heterocycle, optionally substituted heteroaryl, —(CH₂)ₘC(O)OR⁴—(CH₂)ₘC(O)SR⁴, —(CH₂)ₘC(O)NHR⁴, —(CH₂)ₘC(O)N(R⁴)₂, —C(O)OR⁴, —C(O)SR⁴, —O(R⁴), —O(aralkyl), —S(R⁴), NO₂, —NR⁴R⁵, —NH(aralkyl), azido, cyano, SCN, OCN, NCO or halo;
each m is independently 1 or 2; and
alternatively, R⁶ and R¹⁰, R⁷ and R⁹, R⁸ and R⁷ or R⁹ and R¹¹ can come together to form a bridged compound selected from the group consisting of optionally substituted carbocycle or optionally substituted heterocycle; or
alternatively, R⁶ and R⁷ or R⁹ and R¹⁰ can come together to form a spiro compound selected from the group consisting of optionally substituted carbocycle or optionally substituted heterocycle; and
each W is independently O, S or CH.

2. The compound of claim 1 wherein the compound is of Formula I.

3. The compound of claim 1 wherein W is O.

4. The compound of claim 1 wherein each B is independently H or a straight chained, branched or cyclic optionally substituted alkyl.

5. The compound of claim 3 wherein each B is H.

6. The compound of claim 1 wherein R⁷ and R⁹ are independently OH or OR².

7. The compound of claim 1 wherein R¹ is H or phosphate.

8. The compound of claim 1 wherein A is CONHR⁴.

9. A compound of Formula (III), (IV) or (V), 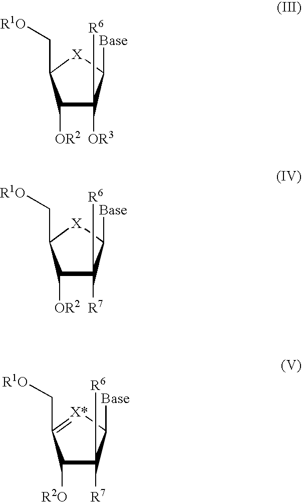
or a pharmaceutically acceptable salt or ester thereof, wherein:
R¹, R² and R³ are each independently H, optionally substituted alkyl; acyl; phosphate; sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹, R² or R³ is independently H or phosphate; wherein in one embodiment R² and/or R³ is not phosphate;
each R⁶ is independently H, OH, NO₂, halo, azido, alkenyl and alkynyl an optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, —(CH₂)ₘC(O)N(R⁴)₂, C(O)OR⁴ or cyano;
X and X* are independently O or CH;
each R⁷ is independently OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle, optionally substituted heterocycle, optionally substituted heteroaryl, —(CH₂)ₘC(O)OR⁴, (CH₂)ₘC(O)SR⁴—(CH₂)ₘC(O)NHR⁴, (CH₂)ₘC(O)N(4)₂, —C(O)OR⁴, —C(O)SR⁴, —O(R⁴), —S(4), NO₂, —NR⁴R⁵, azido, cyano, SCN, OCN, NCO or halo; and
alternatively, R⁶ and R⁷ can come together to form a spiro compound selected from the group consisting of optionally substituted carbocycle or optionally substituted heterocycle;
each m is independently 1 or 2;
and Base is independently: 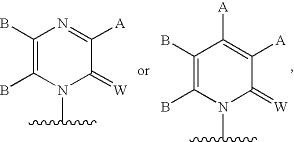
wherein
each A is independently a straight, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, SH, —S-alkyl, —S-acyl, —S-aryl, —S-cycloalkyl, —S-aralkyl, —CO₂-alkyl, —CONH-alkyl, —CON-dialkyl, CF₃, —CHₘOH, —(CH₂)ₘNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each B is independently H, a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, —CO—O-alkoxyalkyl, —CONHR⁴, —C(NR⁴)N(R⁴)₂, —C(S)N(R⁴)₂, —CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, —O-alkenyl, —O-alkynyl, —O-aryl, —O-aralkyl, —O-acyl, —O-cycloalkyl, —NH-alkyl, —N-dialkyl, —NH-acyl, —NH-aryl, —NH-aralkyl, —NH-cycloalkyl, SH, —S-alkyl, —S-acyl, —S-aryl, —S-cycloalkyl, —S-aralkyl, —CO₂-alkyl, —CONH-alkyl, —CON-dialkyl, CF₃, —CHₘOH, —(CH₂)ₘNH₂, —(CH₂)ₘC(O)OH, —(CH₂)ₘCN, —(CH₂)ₘNO₂, —(CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy; and
each W is independently O, S or CH.

10. A compound of Formula (VI), 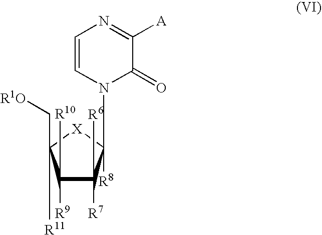
or a pharmaceutically acceptable salt or ester thereof, wherein:
R¹ is independently H, optionally substituted alkyl; acyl; phosphate; sulfonate ester including optionally substituted alkyl or arylalkyl sulfonyl including methanesulfonyl and benzyl, wherein the phenyl group is optionally substituted with one or more substituents as described in the definition of aryl given herein; a lipid, including a phospholipid; an amino acid; a carbohydrate; a peptide; cholesterol; or other pharmaceutically acceptable leaving group which when administered in vivo is capable of providing a compound wherein R¹ is independently H or phosphate;
each A is independently a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONHR⁴, C(NR⁴)N(R⁴)₂, C(S)N(R⁴)₂, CON(R⁴)₂, chloro, bromo, fluoro, iodo, CN, N3, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, O-alkenyl, O-alkynyl, O-aryl, O-aralkyl, —O-acyl, O-cycloalkyl, NH-alkyl, N-dialkyl, NH-acyl, N-aryl, N-aralkyl, NH-cycloalkyl, SH, S-alkyl, S-acyl, S-aryl, S-cycloalkyl, S-aralkyl, CO₂-alkyl, CONH-alkyl, CON-dialkyl, CF₃, CHₘOH, (CH₂)ₘNH₂, (CH₂₋)ₘC(O)OH, (CH₂)ₘCN, (CH₂)ₘNO₂ (CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each B is independently H, a straight chained, branched or cyclic optionally substituted alkyl, CH₃, CF₃, C(Y³)₃, 2-Br-ethyl, CH₂F, CH₂Cl, CH₂CF₃, CF₂CF₃, C(Y³)₂C(Y³)₃, CH₂OH, optionally substituted alkenyl, optionally substituted alkynyl, COOR⁴, COO-aryl, CO—O-alkoxyalkyl, CONFR⁴, C(NR⁴)N(R⁴)₂, C(S)N(R⁴)₂, CON(R⁴)₂, chloro, bromo, fluoro, iodo, CN, N₃, OH, OR⁴, NH₂, NHR⁴, NR⁴R⁵, SH or SR⁵, —C(═S)NH₂, —C(═NH)NH₂, —C-(5-membered heterocycle) having one or more O, S or N atoms, —C-imidazole; cycloalkyl, acyl, Br-vinyl, —O-alkyl, O-alkenyl, O-alkynyl, O-aryl, O-aralkyl, —O-acyl, O-cycloalkyl, NH-alkyl, N-dialkyl, NH-acyl, N-aryl, N-aralkyl, NH-cycloalkyl, SH, S-alkyl, S-acyl, S-aryl, S-cycloalkyl, S-aralkyl, CO₂-alkyl, CONH-alkyl, CON-dialkyl, CF₃, CHₘOH, (CH₂)ₘNH₂, (CH₂₋)ₘC(O)OH, (CH₂)ₙCN, (CH₂)ₘNO₂ (CH₂)ₘCONH₂, C₁₋₄ alkylamino, di(C₁₋₄ alkyl)amino, C₃₋₆ cycloalkylamino, C₁₋₄ alkoxycarbonyl, N₃ or C₁₋₆ alkoxy;
each Y³ is independently H, F, Cl, Br or I;
each R⁴ and R⁵ is independently hydrogen, acyl, alkyl, lower alkyl, alkenyl, alkynyl or cycloalkyl.
X is O or CH;
each R⁶ is independently an optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, —(CH₂)ₘC(O)OR⁴, —(CH₂)ₘC(O)NHR⁴, (CH₂)ₘC(O)N(R⁴)₂, C(O)OR⁴ or cyano;
each R⁷ is independently OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, haloalkenyl, Br-vinyl, optionally substituted alkynyl, haloalkynyl, optionally substituted carbocycle, optionally substituted heterocycle, optionally substituted heteroaryl, —(CH₂)ₘC(O)OR⁴, —(CH₂)ₘC(O)SR⁴—(CH₂)ₘC(O)NHR⁴, (CH₂)ₘC(O)N(R⁴)₂, —C(O)OR⁴, —C(O)SR⁴, —O(R⁴), —S(R⁴), NO₂, —NR⁴R⁵, azido, cyano, SCN, OCN, NCO or halo;
each R⁸ and R¹¹ is independently hydrogen, an optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, haloalkenyl, Br-vinyl, optionally substituted alkynyl, haloalkynyl, —CH₂C(O)N(R⁴)₂, —(CH₂)ₘC(O)OR⁴, —(CH₂)ₘC(O)NHR⁴, —C(O)OR⁴, cyano, NH-acyl or N(acyl)₂;
each R⁹ and R¹⁰ are independently hydrogen, OH, OR², optionally substituted alkyl, CH₃, CH₂CN, CH₂N₃, CH₂NH₂, CH₂NHCH₃, CH₂N(CH₃)₂, CH₂OH, halogenated alkyl, CF₃, C(Y³)₃, 2-Br-ethyl, CH₂F, CH₂Cl, CH₂CF₃, CF₂CF₃, C(Y³)₂C(Y³)₃, optionally substituted alkenyl, alkenyl, alkynyl, NO₂, haloalkenyl, Br-vinyl, optionally substituted alkynyl, haloalkynyl, optionally substituted carbocycle, optionally substituted heterocycle, optionally substituted heteroaryl, —(CH₂)ₘC(O)OR⁴—(CH₂)ₘC(O)SR⁴, —(CH₂)ₘC(O)NHR⁴, —(CH₂)ₘC(O)N(R⁴)₂, —C(O)OR⁴, —C(O)SR⁴, —O(R), —O(aralkyl), —S(R⁴), NO₂, —NR⁴R⁵, —NH(aralkyl), azido, cyano, SCN, OCN, NCO or halo;
each m is independently 1 or 2; and
alternatively, R⁶ and R¹⁰, R⁷ and R⁹, R⁸ and R⁷ or R⁹ and R¹¹ can come together to form a bridged compound selected from the group consisting of optionally substituted carbocycle or optionally substituted heterocycle; or
alternatively, R⁶ and R⁷ or R⁹ and R¹⁰ can come together to form a spiro compound selected from the group consisting of optionally substituted carbocycle or optionally substituted heterocycle.

11. The compound of claim 10 wherein X is O.

12. The compound of claim 10 wherein each R⁶ is independently an optionally substituted lower alkyl, optionally substituted alkenyl, optionally substituted alkynyl, optionally substituted cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃.

13. The compound of claim 10 wherein each R⁷ is independently —OH, optionally substituted lower alkyl, optionally substituted alkenyl, optionally substituted alkynyl, optionally substituted cycloalkyl, —O-alkyl, —O-alkenyl, —O-alkynyl, —O-aralkyl, —O-cycloalkyl-, O-acyl, F, Cl, Br, I, CN, NC, SCN, OCN, NCO, NO₂, NH₂, N₃, NH-acyl, NH-alkyl, N-dialkyl, NH-alkenyl, NH-alkynyl, NH-aralkyl, NH-cycloalkyl, SH, S-alkyl, S-alkenyl, S-alkynyl, S-aralkyl, S-acyl, S-cycloalkyl, CO₂-alkyl, CONH-alkyl, CON-dialkyl, CONH-alkenyl, CONH-alkynyl, CONH-aralkyl, CONH-cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH₂, an optionally substituted 3-7 membered carbocyclic, and an optionally substituted 3-7 membered heterocyclic ring having O, S and/or N independently as a heteroatom taken alone or in combination.

14. The compound of claim 10 wherein each R⁹ is independently hydrogen, optionally substituted lower alkyl, optionally substituted alkenyl, optionally substituted alkynyl, optionally substituted cycloalkyl, —OH, —O-alkyl, —O-alkenyl, —O-alkynyl, —O-aralkyl, —O-cycloalkyl-, O-acyl, F, Cl, Br, I, CN, NC, SCN, OCN, NCO, NO₂, NH₂, N₃, NH-acyl, NH-alkyl, N-dialkyl, NH-alkenyl, NH-alkynyl, NH-aralkyl, NH-cycloalkyl, SH, S-alkyl, S-alkenyl, S-alkynyl, S-aralkyl, S-acyl, S-cycloalkyl, CO₂-alkyl, CONH-alkyl, CON-dialkyl, CONH-alkenyl, CONH-alkynyl, CONH-aralkyl, CONH-cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH₂, an optionally substituted 3-7 membered carbocyclic, and an optionally substituted 3-7 membered heterocyclic ring having O, S and/or N independently as a heteroatom taken alone or in combination.

15. The compound of claim 10 wherein each R¹⁰ is independently hydrogen, an optionally substituted lower alkyl, optionally substituted alkenyl, optionally substituted alkynyl, optionally substituted cycloalkyl, CH₂OH, CH₂NH₂, CH₂NHCH₃, CH₂N(CH₃)₂, CH₂F, CH₂Cl, CH₂N₃, CH₂CN, CH₂CF₃, CF₃, CF₂CF₃, (CH₂)ₘCOOH, (CH₂)ₘCONH.

16. The compound of claim 10 wherein each R⁸ and R¹¹ is independently H, CH₃, CH₂OH, CH₂F, CH₂N₃, (CH₂)ₘCOOH, (CH₂)ₘCONH₂, and N-acyl.

17. The compound of claim 10 wherein A is CONH₂.

18. The compound of claim 10 wherein each m is independently 1.

19. A method for the treatment or propylaxis of a host infected with a flavivirus, pestivirus or hepacivirus infection comprising administering to the host an effective treatment amount of a compound of claim 1, 9 or 10, optionally in a pharmaceutically acceptable carrier.

20. The method of claim 19, wherein the infection is an HCV infection.

21. The method of claim 19, wherein the infection is not an HCV infection.

22. The method of claim 19 wherein the host is at risk of being infected with a flavivirus, pestivirus or hepacivirus.

23. The method of claim 19, further comprising administering at least a second antiviral agent.

24. The method of claim 23 wherein the second antiviral agent is selected from the group consisting of Interferon; Ribavirin; Protease inhibitors (such as Substrate-based NS3 protease inhibitors, Non-substrate-based inhibitors, Phenanthrenequinones possessing activity against protease and Selective NS3 inhibitors); Thiazolidine derivatives; Thiazolidines and benzanilides; Helicase; Polymerase inhibitors; Antisense phosphorothioate oligodeoxynucleotides; Inhibitors of IRES-dependent translation; Nuclease-resistant ribozymes; Nucleoside analogs; 1-amino-alkylcyclohexanes; alkyl lipids; vitamin E and other antioxidants; squalene; amantadine; bile acids; N-(phosphonoacetyl)-L-aspartic acid; benzenedicarboxamides; polyadenylic acid derivatives; 2′,3′-dideoxyinosine; benzimidazoles; plant extracts; and piperidenes.

25. A pharmaceutical composition for the treatment of a host infected with a flavivirus, pestivirus or hepacivirus infection comprising a treatment effective amount of a compound of claim 1, 9 or 10, or a pharmaceutically acceptable salt or ester thereof, and a pharmaceutically acceptable carrier.

26. The composition of claim 25 further comprising at least a second antiviral agent.

27. The composition of claim 26 wherein the second antiviral agent is selected from the group consisting of Interferon; Ribavirin; Protease inhibitors (such as Substrate-based NS3 protease inhibitors, Non-substrate-based inhibitors, Phenanthrenequinones possessing activity against protease and Selective NS3 inhibitors); Thiazolidine derivatives; Thiazolidines and benzanilides; Helicase; Polymerase inhibitors; Antisense phosphorothioate oligodeoxynucleotides; Inhibitors of IRES-dependent translation; Nuclease-resistant ribozymes; Nucleoside analogs; 1-amino-alkylcyclohexanes; alkyl lipids; vitamin E and other antioxidants; squalene; amantadine; bile acids; N-(phosphonoacetyl)-L-aspartic acid; benzenedicarboxamides; polyadenylic acid derivatives; 2′,3′-dideoxyinosine; benzimidazoles; plant extracts; and piperidenes.
